(19) United States
(12) Patent Application Publication
(10) Pub. No.: US 2020/0087729 A1
(43) Pub. Date: Mar. 19, 2020

(54) Compositions and Methods to Detect Kidney Fibrosis

(71) Applicants: Walter Joseph Jessen, New Palestine, IN (US); Jennifer Lynn Ennis, Chicago, IL (US); John Robert Asplin, Chicago, IL (US)

(72) Inventors: Walter Joseph Jessen, New Palestine, IN (US); Jennifer Lynn Ennis, Chicago, IL (US); John Robert Asplin, Chicago, IL (US)

(21) Appl. No.: 16/409,520

(22) Filed: May 10, 2019

Related U.S. Application Data
(60) Provisional application No. 62/670,344, filed on May 11, 2018.

(51) Int. Cl. C12Q 1/6883; G01N 33/68; C12Q 1/37

(52) U.S. Cl. CPC C12Q 1/6883; G01N 33/6893; C12Q 1/37; G01N 2800/50; C12Q 2600/156; C12Q 2600/158; G01N 33/6869

(57) ABSTRACT

Disclosed are compositions and methods to detect proteins associated with kidney fibrosis. Such biomarkers may be useful to allow individuals susceptible to kidney fibrosis to manage their lifestyle and reduce further progression of disease.

### RELATED APPLICATIONS

This application claims priority to U.S. Provisional Patent Application No. 62/670,344, filed May 11, 2018. The disclosure of U.S. Provisional Patent Application No. 62/670,344 is incorporated by reference in its entirety herein.

### FIELD

The disclosure relates to methods and compositions for diagnosing kidney fibrosis.

### BACKGROUND

The biological mechanisms behind kidney fibrosis are largely unknown and there are few, if any, biomarkers that provide a reliable indication of this condition. It would be helpful for individuals having susceptibility to kidney fibrosis to adjust their lifestyle so as to avoid triggering an onset of symptoms and/or promoting further progression of the disease. Thus, there is a need to develop and evaluate biomarkers for kidney fibrosis.

### SUMMARY

The present disclosure may be embodied in a variety of ways.

In one embodiment, disclosed is a method to detect biomarkers associated with kidney fibrosis in an individual comprising the steps of: obtaining a biological sample from the individual; and measuring in the biological sample, the amount of the biomarker, and/or the amount or a mutation in a nucleic acid (e.g. genomic DNA or mRNA) that encodes for, or regulates expression of the biomarker. In an embodiment, the biomarker comprises of at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), NPHS1 nephrin (NPHS1), podicin (NPHS2), nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA). In certain embodiments, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2). In certain embodiments, the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7). In certain embodiments, the biomarker comprises at least one of transforming growth factor beta 1 (TGFB1) or metallopeptidase 2 (MMP2).

Additionally and/or alternatively, the method may include measurement of at least one normalization (e.g., housekeeping gene). Or, measurement of various combinations of these biomarkers may be performed.

Other features, objects, and advantages of the disclosure herein are apparent in the detailed description, drawings and claims that follow. It should be understood, however, that the detailed description, the drawings, and the claims, while indicating embodiments of the disclosed methods, compositions and systems, are given by way of illustration only, not limitation. Various changes and modifications within the scope of the invention will become apparent to those skilled in the art.

### FIGURES

The disclosure may be better understood in view of the following non-limiting figures.

FIG. 1 shows an example of a multi-node interaction network identifying biomarkers associated with kidney fibrosis.

FIG. 2 shows an alternate example of a multi-node interaction network identifying biomarkers associated with kidney fibrosis.

FIG. 3 shows an example of biomarkers indicated by data mining as associated with kidney fibrosis.

FIG. 4 shows an example of a system for performing the methods and/or using the compositions disclosed herein.

### DETAILED DESCRIPTION

### Terms and Definitions

In order for the disclosure to be more readily understood, certain terms are first defined. Additional definitions for the following terms and other terms are set forth throughout the specification.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “and/or” generally is used to refer to at least one or the other. In some case the term “and/or” is used interchangeably with the term “or.” The term “including” is used herein to mean, and is used interchangeably with, the phrase “including but not limited to.” The term “such as” is used herein to mean, and is used interchangeably with, the phrase “such as but not limited to.”

Unless defined otherwise, all technical and scientific terms used herein have the same meaning as commonly understood by one of ordinary skill in the art.

Also as used herein, “at least one” contemplates any number from 1 to the entire group. For example, for a listing of four biomarkers, the phrase at “least one” is understood to mean 1, 2, 3 or 4 biomarkers. Similarly, for a listing of 10 biomarkers, the phrase “at least five” is understood to mean 5, 6, 7, 8, 9, or 10 biomarkers.

Also, as used herein, “comprising” includes embodiments more particularly defined using the term “consisting of”

Antibody: As used herein, the term “antibody” refers to a polypeptide consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are typically classified as either kappa or lambda. Heavy chains are typically classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains respectively. An antibody can be specific for a particular antigen. The antibody or its antigen can be either an analyte or a binding partner. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region. While various antibody fragments are defined in terms of the digestion of an intact antibody, one of ordinary skill in the art will appreciate that such Fab′ fragments may be synthesized de novo either chemically or by utilizing recombinant DNA methodology. Thus, the term “antibody,” as used herein also includes antibody fragments either produced by the modification of whole antibodies or synthesized de novo using recombinant DNA methodologies. In some embodiments, antibodies are single chain antibodies, such as single chain Fv (scFv) antibodies in which a variable heavy and a variable light chain are joined together (directly or through a peptide linker) to form a continuous polypeptide. A single chain Fv (“scFv”) polypeptide is a covalently linked VH::VL heterodimer which may be expressed from a nucleic acid including VH- and VL-encoding sequences either joined directly or joined by a peptide-encoding linker. (See, e.g., Huston, et al. (1988) Proc. Nat. Acad. Sci. USA, 85:5879-5883) A number of structures exist for converting the naturally aggregated, but chemically separated light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778.

The term “antibody” includes monoclonal antibodies, polyclonal antibodies, synthetic antibodies and chimeric antibodies, e.g., generated by combinatorial mutagenesis and phage display. The term “antibody” also includes mimetics or peptidomimetics of antibodies. Peptidomimetics are compounds based on, or derived from, peptides and proteins. The peptidomimetics of the present disclosure typically can be obtained by structural modification of a known peptide sequence using unnatural amino acids, conformational restraints, isosteric replacement, and the like.

Allele: As used herein, the term “allele” refers to different versions of a nucleotide sequence of a same genetic locus (e.g., a gene).

Allele specific primer extension (ASPE): As used herein, the term “allele specific primer extension (ASPE)” refers to a mutation detection method utilizing primers which hybridize to a corresponding DNA sequence and which are extended depending on the successful hybridization of the 3′ terminal nucleotide of such primer. Typically, extension primers that possess a 3′ terminal nucleotide which form a perfect match with the target sequence are extended to form extension products. Modified nucleotides can be incorporated into the extension product, such nucleotides effectively labeling the extension products for detection purposes. Alternatively, an extension primer may instead comprise a 3′ terminal nucleotide which forms a mismatch with the target sequence. In this instance, primer extension does not occur unless the polymerase used for extension inadvertently possesses exonuclease activity.

Amplification: As used herein, the term “amplification” refers to any methods known in the art for copying a target nucleic acid, thereby increasing the number of copies of a selected nucleic acid sequence. Amplification may be exponential or linear. A target nucleic acid may be either DNA or RNA. Typically, the sequences amplified in this manner form an “amplicon.” Amplification may be accomplished with various methods including, but not limited to, the polymerase chain reaction (“PCR”), transcription-based amplification, isothermal amplification, rolling circle amplification, etc. Amplification may be performed with relatively similar amount of each primer of a primer pair to generate a double stranded amplicon. However, asymmetric PCR may be used to amplify predominantly or exclusively a single stranded product as is well known in the art (e.g., Poddar, Molec. And Cell. Probes 14:25-32 (2000)). This can be achieved using each pair of primers by reducing the concentration of one primer significantly relative to the other primer of the pair (e.g., 100 fold difference). Amplification by asymmetric PCR is generally linear. A skilled artisan will understand that different amplification methods may be used together.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Also, throughout this application, the term “about” is used to indicate that a value includes the inherent variation of error for the device, the method being employed to determine the value, or the variation that exists among samples.

Associated with a syndrome or disease of interest: As used herein, “associated with a syndrome or disease of interest” means that the variant is found with in patients with the syndrome or disease of interest more than in non-syndromic or non-disease controls. Generally, the statistical significance of such association can be determined by assaying a plurality of patients.

Biological Sample and Sample: As used herein, the term “sample” or “biological sample” encompasses any sample obtained from a subject, an individual or other biological source. A biological sample can, by way of non-limiting example, include blood, plasma, serum, liquid or tissue biopsy, cell-free nucleic acid (e.g., DNA or RNA), urine, feces, epidermal sample, skin sample, cheek swab, sperm, amniotic fluid, cultured cells, bone marrow sample and/or chorionic villi. Convenient biological samples may be obtained by, for example, scraping cells from the surface of the buccal cavity. The term biological sample encompasses samples which have been processed to release or otherwise make available a nucleic acid or protein for detection as described herein. Also included is cell-free DNA such as that found in plasma, amniotic fluid and the like. For example, a biological sample may include a cDNA that has been obtained by reverse transcription of RNA from cells in a biological sample. The biological sample may be obtained from a stage of life such as a fetus, young adult, adult, and the like. Fixed or frozen tissues also may be used.

Biomarker: As used herein, the term “biomarker” or “marker” refers to one or more nucleic acids, polypeptides and/or other biomolecules (e.g., cholesterol, lipids) that can be used to diagnose, or to aid in the diagnosis or prognosis of a disease or syndrome of interest, either alone or in combination with other biomarkers; monitor the progression of a disease or syndrome of interest; and/or monitor the effectiveness of a treatment for a syndrome or a disease of interest. As used herein, and as is commonly employed by those of skill in the art, biomarkers are listed as either the hormone or other small molecule (e.g., corticosterone, aldosterone), protein (epidermal growth factor receptor), or gene (EGFR or EGFR) that encodes the biomarker.

Binding agent: As used herein, the term “binding agent” refers to a molecule that can specifically and selectively bind to a second (i.e., different) molecule of interest. The interaction may be non-covalent, for example, as a result of hydrogen-bonding, van der Waals interactions, or electrostatic or hydrophobic interactions, or it may be covalent. The term “soluble binding agent” refers to a binding agent that is not associated with (i.e., covalently or non-covalently bound) to a solid support.

Carrier: The term “carrier” refers to a person who is symptom-free but carries a mutation that can be passed to his/her children. Typically, for an autosomal recessive disorder, a carrier has one allele that contains a disease causing mutation and a second allele that is normal or not disease-related.

Chromatography: The term “chromatography” refers to a process in which a chemical mixture carried by a liquid or gas is separated into components as a result of differential distribution of the chemical entities as they flow around or over a stationary liquid or solid phase. The term, “liquid chromatography” (LC) means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). “Liquid chromatography” includes reverse phase liquid chromatography (RPLC), high performance liquid chromatography (HPLC) and high turbulence liquid chromatography (HTLC). The term “HPLC” or “high performance liquid chromatography” refers to liquid chromatography in which the degree of separation is increased by forcing the mobile phase under high pressure through a stationary phase, typically a densely packed column.

Coding sequence vs. non-coding sequence: As used herein, the term “coding sequence” refers to a sequence of a nucleic acid or its complement, or a part thereof, that can be transcribed and/or translated to produce the mRNA for and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the encoding sequence can be deduced therefrom. As used herein, the term “non-coding sequence” refers to a sequence of a nucleic acid or its complement, or a part thereof, that is not transcribed into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, etc.

Complement: As used herein, the terms “complement,” “complementary” and “complementarity,” refer to the pairing of nucleotide sequences according to Watson/Crick pairing rules. For example, a sequence 5′-GCGGTCCCA-3′ has the complementary sequence of 5′-TGGGACCGC-3′. A complement sequence can also be a sequence of RNA complementary to the DNA sequence. Certain bases not commonly found in natural nucleic acids may be included in the complementary nucleic acids including, but not limited to, inosine, 7- deazaguanine, Locked Nucleic Acids (LNA), and Peptide Nucleic Acids (PNA). Complementary need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Conserved: As used herein, the term “conserved residues” refers to amino acids that are the same among a plurality of proteins having the same structure and/or function. A region of conserved residues may be important for protein structure or function. Thus, contiguous conserved residues as identified in a three-dimensional protein may be important for protein structure or function. To find conserved residues, or conserved regions of 3-D structure, a comparison of sequences for the same or similar proteins from different species, or of individuals of the same species, may be made.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control containing a known amount of a biomarker of interest or a negative control that does not contain any of the biomarker of interest.

In some embodiments, a “control” or “predetermined standard” for a biomarker refers to the levels of expression of the biomarker in healthy subjects or the expression levels of said biomarker in non-diseased or non-syndromic tissue from the same subject. The control or predetermined standard expression levels or amounts of protein for a given biomarker can be established by prospective and/or retrospective statistical studies using only routine experimentation. Such predetermined standard expression levels and/or protein levels (amounts) can be determined by a person having ordinary skill in the art using well known methods.

Crude: As used herein, the term “crude,” when used in connection with a biological sample, refers to a sample which is in a substantially unrefined state. For example, a crude sample can be cell lysates or biopsy tissue sample. A crude sample may exist in solution or as a dry preparation.

Deletion: As used herein, the term “deletion” encompasses a mutation that removes one or more nucleotides from a naturally-occurring nucleic acid.

Disease or syndrome of interest: As used herein, a disease or syndrome of interest is kidney fibrosis.

Detect: As used herein, the term “detect”, “detected” or “detecting” includes “measure,” “measured” or” measuring” and vice versa.

Detectable moiety: As used herein, the term “detectable moiety” or “detectable biomolecule” or “reporter” refers to a molecule that can be measured in a quantitative assay. For example, a detectable moiety may comprise an enzyme that may be used to convert a substrate to a product that can be measured (e.g., a visible product). Or, a detectable moiety may be a radioisotope that can be quantified. Or, a detectable moiety may be a fluorophore. Or, a detectable moiety may be a luminescent molecule. Or, other detectable molecules may be used.

Epigenetic: As used herein, an epigenetic element can change gene expression by a mechanism other than a change in the underlying DNA sequences. Such elements may include elements that regulate paramutation, imprinting, gene silencing, X chromosome inactivation, position effect, reprogramming, transvection, maternal effects, histone modification, and heterochromatin.

Epitope: As used herein, the term “epitope” refers to a fragment or portion of a molecule or a molecule compound (e.g., a polypeptide or a protein complex) that makes contact with a particular antibody or antibody like proteins.

Exon: As used herein an exon is a nucleic acid sequence that is found in mature or processed RNA after other portions of the RNA (e.g., intervening regions known as introns) have been removed by RNA splicing. As such, exon sequences generally encode for proteins or portions of proteins. An intron is the portion of the RNA that is removed from surrounding exon sequences by RNA splicing.

Expression and expressed RNA: As used herein expressed RNA is an RNA that encodes for a protein or polypeptide (“coding RNA”), and any other RNA that is transcribed but not translated (“non-coding RNA”). The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

The measurement of an amount of a protein and/or the expression of a biomarker of the disclosure may be assessed by any of a wide variety of well-known methods for detecting expression of a transcribed molecule or its corresponding protein. Non-limiting examples of such methods include immunological methods for detection of secreted proteins, protein purification methods, protein function or activity assays, nucleic acid hybridization methods, nucleic acid reverse transcription methods, and nucleic acid amplification methods. In certain embodiments, expression of a biomarker gene is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a protein corresponding to the biomarker gene, such as the protein encoded by the open reading frame corresponding to the biomarker gene or such a protein which has undergone all or a portion of its normal post-translational modification. In certain embodiments, a reagent may be directly or indirectly labeled with a detectable substance. The detectable substance may be, for example, selected, e.g., from a group consisting of radioisotopes, fluorescent compounds, enzymes, and enzyme co-factor. Methods of labeling antibodies are well known in the art.

In another embodiment, expression of a biomarker gene is assessed by preparing mRNA/cDNA (i.e. a transcribed polynucleotide) from cells in a sample, and by hybridizing the mRNA/cDNA with a reference polynucleotide which is a complement of a polynucleotide comprising the biomarker gene, and fragments thereof cDNA can, optionally, be amplified using any of a variety of polymerase chain reaction methods prior to hybridization with the reference polynucleotide; preferably, it is not amplified.

Familial history: As used herein, the term “familial history” typically refers to occurrence of events (e.g., disease related disorder or mutation carrier) relating to an individual's immediate family members including parents and siblings. Family history may also include grandparents and other relatives.

Flanking: As used herein, the term “flanking” is meant that a primer hybridizes to a target nucleic acid adjoining a region of interest sought to be amplified on the target. The skilled artisan will understand that preferred primers are pairs of primers that hybridize 5′ from a region of interest, one on each strand of a target double stranded DNA molecule, such that nucleotides may be add to the 3′ end of the primer by a suitable DNA polymerase. For example, primers that flank mutant sequences do not actually anneal to the mutant sequence but rather anneal to a sequence that adjoins the mutant sequence. In some cases, primers that flank an exon are generally designed not to anneal to the exon sequence but rather to anneal to sequence that adjoins the exon (e.g. intron sequence). However, in some cases, amplification primer may be designed to anneal to the exon sequence.

Gene: As used herein a gene is a unit of heredity. Generally, a gene is a portion of DNA that encodes a protein or a functional RNA. A gene is a locatable region of genomic sequence corresponding to a unit of inheritance. A gene may be associated with regulatory regions, transcribed regions, and or other functional sequence regions.

Genotype: As used herein, the term “genotype” refers to the genetic constitution of an organism. More specifically, the term refers to the identity of alleles present in an individual. “Genotyping” of an individual or a DNA sample refers to identifying the nature, in terms of nucleotide base, of the two alleles possessed by an individual at a known polymorphic site.

Gene regulatory element: As used herein a gene regulatory element or regulatory sequence is a segment of DNA where regulatory proteins, such as transcription factors, bind to regulate gene expression. Such regulatory regions are often upstream of the gene being regulated.

Healthy individual: As used herein, the term “healthy individual” or “control” refers to a subject has not been diagnosed with the syndrome and/or disease of interest.

Heterozygous: As used herein, the term “heterozygous” or “HET” refers to an individual possessing two different alleles of the same gene. As used herein, the term “heterozygous” encompasses “compound heterozygous” or “compound heterozygous mutant.” As used herein, the term “compound heterozygous” refers to an individual possessing two different alleles. As used herein, the term “compound heterozygous mutant” refers to an individual possessing two different copies of an allele, such alleles are characterized as mutant forms of a gene.

Homozygous: As used herein, the term “homozygous” refers to an individual possessing two copies of the same allele. As used herein, the term “homozygous mutant” refers to an individual possessing two copies of the same allele, such allele being characterized as the mutant form of a gene.

Hybridize: As used herein, the term “hybridize” or “hybridization” refers to a process where two complementary nucleic acid strands anneal to each other under appropriately stringent conditions. Oligonucleotides or probes suitable for hybridizations typically contain 10-100 nucleotides in length (e.g., 18-50, 12-70, 10-30, 10-24, 18-36 nucleotides in length). Nucleic acid hybridization techniques are well known in the art. Those skilled in the art understand how to estimate and adjust the stringency of hybridization conditions such that sequences having at least a desired level of complementary will stably hybridize, while those having lower complementary will not.

For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J. Identity or percent identical: As used herein, the terms “identity” or “percent identical” refers to sequence identity between two amino acid sequences or between two nucleic acid sequences. Percent identity can be determined by aligning two sequences and refers to the number of identical residues (i.e., amino acid or nucleotide) at positions shared by the compared sequences. Sequence alignment and comparison may be conducted using the algorithms standard in the art (e.g. Smith and Waterman, 1981, Adv. Appl. Math. 2:482; Needleman and Wunsch, 1970, J. Mol. Biol. 48:443; Pearson and Lipman, 1988, Proc. Natl. Acad. Sci., USA, 85:2444) or by computerized versions of these algorithms (Wisconsin Genetics Software Package Release 7.0, Genetics Computer Group, 575 Science Drive, Madison, Wis.) publicly available as BLAST and FASTA. Also, ENTREZ, available through the National Institutes of Health, Bethesda Md., may be used for sequence comparison. In other cases, commercially available software, such as GenomeQuest, may be used to determine percent identity. When utilizing BLAST and Gapped BLAST programs, the default parameters of the respective programs (e.g., BLASTN; available at the Internet site for the National Center for Biotechnology Information) may be used. In one embodiment, the percent identity of two sequences may be determined using GCG with a gap weight of 1, such that each amino acid gap is weighted as if it were a single amino acid mismatch between the two sequences. Or, the ALIGN program (version 2.0), which is part of the GCG (Accelrys, San Diego, Calif.) sequence alignment software package may be used.

As used herein, the term at least 90% identical thereto includes sequences that range from 90 to 100% identity to the indicated sequences and includes all ranges in between. Thus, the term at least 90% identical thereto includes sequences that are 91, 91.5, 92, 92.5, 93, 93.5, 94, 94.5, 95, 95.5, 96, 96.5, 97, 97.5, 98, 98.5, 99, 99.5 or 100 percent identical to the indicated sequence. Similarly, the term “at least 70% identical includes sequences that range from 70 to 100% identical, with all ranges in between. The determination of percent identity is determined using the algorithms described herein.

Insertion or addition: As used herein, the term “insertion” or “addition” refers to a change in an amino acid or nucleotide sequence resulting in the addition of one or more amino acid residues or nucleotides, respectively, as compared to the naturally occurring molecule.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism such as a human or a non-human animal.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Labeled: The terms “labeled” and “labeled with a detectable agent or moiety” are used herein interchangeably to specify that an entity (e.g., a nucleic acid probe, antibody, etc.) can be measured by detection of the label (e.g., visualized, detection of radioactivity and the like) for example following binding to another entity (e.g., a nucleic acid, polypeptide, etc.). The detectable agent or moiety may be selected such that it generates a signal which can be measured and whose intensity is related to (e.g., proportional to) the amount of bound entity. A wide variety of systems for labeling and/or detecting proteins and peptides are known in the art. Labeled proteins and peptides can be prepared by incorporation of, or conjugation to, a label that is detectable by spectroscopic, photochemical, biochemical, immunochemical, electrical, optical, chemical or other means. A label or labeling moiety may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents include, but are not limited to, radionucleotides, fluorophores, chemiluminescent agents, microparticles, enzymes, colorimetric labels, magnetic labels, haptens, molecular beacons, aptamer beacons, and the like.

Micro RNA: As used herein microRNAs (miRNAs) are short (20-24 nucleotide) non-coding RNAs that are involved in post-transcriptional regulation of gene expression. microRNA can affect both the stability and translation of mRNAs. For example, microRNAs can bind to complementary sequences in the 3′UTR of target mRNAs and cause gene silencing. miRNAs are transcribed by RNA polymerase II as part of capped and polyadenylated primary transcripts (pri-miRNAs) that can be either protein-coding or non-coding. The primary transcript can be cleaved by the Drosha ribonuclease III enzyme to produce an approximately 70-nucleotide stem-loop precursor miRNA (pre-miRNA), which can further be cleaved by the cytoplasmic Dicer ribonuclease to generate the mature miRNA and antisense miRNA star (miRNA*) products. The mature miRNA can be incorporated into a RNA-induced silencing complex (RISC), which can recognize target mRNAs through imperfect base pairing with the miRNA and most commonly results in translational inhibition or destabilization of the target mRNA.

Multiplex PCR: As used herein, the term “multiplex PCR” refers to concurrent amplification of two or more regions which are each primed using a distinct primers pair.

Multiplex ASPE: As used herein, the term “multiplex ASPE” refers to an assay combining multiplex PCR and allele specific primer extension (ASPE) for detecting polymorphisms. Typically, multiplex PCR is used to first amplify regions of DNA that will serve as target sequences for ASPE primers. See the definition of allele specific primer extension.

Mutation and/or variant: As used herein, the terms mutation and variant are used interchangeably to describe a nucleic acid or protein sequence change. The term “mutant” as used herein refers to a mutated, or potentially non-functional form of a gene. The term includes any mutation that renders a gene not functional from a point mutation to large chromosomal rearrangements as is known in the art.

Nucleic acid: As used herein, a “nucleic acid” is a polynucleotide such as deoxyribonucleic acid (DNA) or ribonucleic acid (RNA). The term is used to include single-stranded nucleic acids, double-stranded nucleic acids, mRNA, and RNA and DNA made from nucleotide or nucleoside analogues.

Polypeptide or protein: As used herein, the term “polypeptide” and/or “protein” refers to a polymer of amino acids, and not to a specific length. Thus, peptides, oligopeptides and proteins are included within the definition of polypeptide and/or protein. “Polypeptide” and “protein” are used interchangeably herein to describe protein molecules that may comprise either partial or full-length proteins. The term “peptide” is used to denote a less than full-length protein or a very short protein unless the context indicates otherwise.

As is known in the art, “proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. Typically, the amino acids making up a protein are numbered in order, starting at the amino terminal residue and increasing in the direction toward the carboxy terminal residue of the protein. Abbreviations for amino acid residues are the standard 3-letter and/or 1-letter codes used in the art to refer to one of the 20 common L-amino acids.

As used herein, a polypeptide or protein “domain” comprises a region along a polypeptide or protein that comprises an independent unit. Domains may be defined in terms of structure, sequence and/or biological activity. In one embodiment, a polypeptide domain may comprise a region of a protein that folds in a manner that is substantially independent from the rest of the protein. Domains may be identified using domain databases such as, but not limited to PFAM, PRODOM, PROSITE, BLOCKS, PRINTS, SBASE, ISREC PROFILES, SAMRT, and PROCLASS.

Primer: As used herein, the term “primer” refers to a short single-stranded oligonucleotide capable of hybridizing to a complementary sequence in a nucleic acid sample. Typically, a primer serves as an initiation point for template dependent DNA synthesis. Deoxyribonucleotides can be added to a primer by a DNA polymerase. In some embodiments, such deoxyribonucleotide addition to a primer is also known as primer extension. The term primer, as used herein, includes all forms of primers that may be synthesized including peptide nucleic acid primers, locked nucleic acid primers, phosphorothioate modified primers, labeled primers, and the like. A “primer pair” or “primer set” for a PCR reaction typically refers to a set of primers typically including a “forward primer” and a “reverse primer.” As used herein, a “forward primer” refers to a primer that anneals to the anti-sense strand of dsDNA. A “reverse primer” anneals to the sense-strand of dsDNA.

Polymorphism: As used herein, the term “polymorphism” refers to the coexistence of more than one form of a gene or portion thereof.

Portion and Fragment: As used herein, the terms “portion” and “fragment” are used interchangeably to refer to parts of a polypeptide, nucleic acid, or other molecular construct.

Purify or Separate: The terms “purify” or “separate” or derivations thereof do not necessarily refer to the removal of all materials other than the analyte(s) of interest from a sample matrix. Instead, in some embodiments, the terms “purify” or “separate” refer to a procedure that enriches the amount of one or more analytes of interest relative to one or more other components present in the sample matrix. In some embodiments, a “purification” or “separation” procedure can be used to remove one or more components of a sample that could interfere with the detection of the analyte, for example, one or more components that could interfere with detection of an analyte by mass spectrometry.

Quantitative and Real-Time PCR: The term “quantitative PCR” or “qPCR” is used to describe a method that allows for quantification of the amounts of the target nucleic acid sequence used at the start at the PCR reaction. The term “quantitative PCR” encompasses all PCR-based techniques that allow for quantification of the initially present target nucleic acid sequences.

The term “real-time PCR” is used to describe a method for detecting and measuring products generated during each cycle of a PCR that are proportionate to the amount of template nucleic acid prior to the start of PCR. The information obtained, such as an amplification curve, can be used to determine the presence of a target nucleic acid and/or quantitate the initial amounts of a target nucleic acid sequence. In some examples, real-time PCR is real time reverse transcriptase PCR (rRT-PCR). The term “real-time PCR” is used to denote a subset of quantitative PCR techniques that allow for detection of PCR product throughout the PCR reaction, or in real-time.

Sense strand vs. anti-sense strand: As used herein, the term “sense strand” refers to the strand of double-stranded DNA (dsDNA) that includes at least a portion of a coding sequence of a functional protein. As used herein, the term “anti-sense strand” refers to the strand of dsDNA that is the reverse complement of the sense strand.

Significant difference: As used herein, the term “significant difference” is well within the knowledge of a skilled artisan and will be determined empirically with reference to each particular biomarker. For example, a significant difference in the expression of a biomarker in a subject with the disease or syndrome of interest as compared to a healthy subject is any difference in protein amounts which is statistically significant.

Similar or homologue: As used herein, the term “similar” or “homologue” when referring to amino acid or nucleotide sequences means a polypeptide having a degree of homology or identity with the wild-type amino acid sequence. Homology comparisons can be conducted by eye, or more usually, with the aid of readily available sequence comparison programs. These commercially available computer programs can calculate percent homology between two or more sequences (e.g. Wilbur, W. J. and Lipman, D. J., 1983, Proc. Natl. Acad. Sci. USA, 80:726-730). For example, homologous sequences may be taken to include an amino acid sequences which in alternate embodiments are at least 70% identical, 75% identical, 80% identical, 85% identical, 90% identical, 95% identical, 97% identical, or 98% identical to each other.

Specific: As used herein, the term “specific,” when used in connection with an oligonucleotide primer, refers to an oligonucleotide or primer, which under appropriate hybridization or washing conditions, is capable of hybridizing to the target of interest and not substantially hybridizing to nucleic acids which are not of interest. Higher levels of sequence identity are preferred and include at least 60%, 65%, 70%, 75%, 80%, 85%, 90%, 95%, 98%, 99%, or 100% sequence identity. In some embodiments, a specific oligonucleotide or primer contains at least 4, 6, 8, 10, 12, 14, 16, 18, 20, 22, 24, 26, 28, 30, 35, 40, 45, 50, 55, 60, 65, 70, or more bases of sequence identity with a portion of the nucleic acid to be hybridized or amplified when the oligonucleotide and the nucleic acid are aligned.

As is known in the art, conditions for hybridizing nucleic acid sequences to each other can be described as ranging from low to high stringency. Generally, highly stringent hybridization conditions refer to washing hybrids in low salt buffer at high temperatures. Hybridization may be to filter bound DNA using hybridization solutions standard in the art such as 0.5 M NaHPO₄, 7% sodium dodecyl sulfate (SDS), at 65° C., and washing in 0.25 M NaHPO₄, 3.5% SDS followed by washing 0.1× SSC/0.1% SDS at a temperature ranging from room temperature to 68° C. depending on the length of the probe (see e.g. Ausubel, F. M. et al., Short Protocols in Molecular Biology, 4^{th} Ed., Chapter 2, John Wiley & Sons, N.Y). For example, a high stringency wash comprises washing in 6× SSC/0.05% sodium pyrophosphate at 37° C. for a 14 base oligonucleotide probe, or at 48° C. for a 17 base oligonucleotide probe, or at 55° C. for a 20 base oligonucleotide probe, or at 60° C. for a 25 base oligonucleotide probe, or at 65° C. for a nucleotide probe about 250 nucleotides in length. Nucleic acid probes may be labeled with radionucleotides by end-labeling with, for example, [γ-³²P]ATP, or incorporation of radiolabeled nucleotides such as [α-³²P]dCTP by random primer labeling. Alternatively, probes may be labeled by incorporation of biotinylated or fluorescein labeled nucleotides, and the probe detected using streptavidin or anti-fluorescein antibodies.

siRNA: As used herein, siRNA (small inhibitory RNA) is essentially a double-stranded RNA molecule composed of about 20 complementary nucleotides. siRNA is created by the breakdown of larger double-stranded (ds) RNA molecules. siRNA can suppress gene expression by inherently splitting its corresponding mRNA in two by way of the interaction of the siRNA with the mRNA, leading to degradation of the mRNA. siRNAs can also interact with DNA to facilitate chromatin silencing and the expansion of heterochromatin.

Subject or Individual or Patient: As used herein, the term “subject” or “individual” refers to a human or any non-human animal. A subject or individual can be a patient, which refers to a human presenting to a medical provider for diagnosis or treatment of a disease, and in some cases, wherein the disease is kidney fibrosis or a related syndrome. A human includes pre and post-natal forms. Also, as used herein, the terms “individual,” “subject” or “patient” includes all warm-blooded animals. In one embodiment the subject or individual or patient is a human. In one embodiment, the individual is a subject or patient who has kidney fibrosis or has an enhanced risk of developing kidney fibrosis.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially complementary: As used herein, the term “substantially complementary” refers to two sequences that can hybridize under stringent hybridization conditions. The skilled artisan will understand that substantially complementary sequences need not hybridize along their entire length. In some embodiments, “stringent hybridization conditions” refer to hybridization conditions at least as stringent as the following: hybridization in 50% formamide, 5× SSC, 50 mM NaH₂PO₄, pH 6.8, 0.5% SDS, 0.1 mg/mL sonicated salmon sperm DNA, and 5× Denhart's solution at 42° C. overnight; washing with 2× SSC, 0.1% SDS at 45° C.; and washing with 0.2× SSC, 0.1% SDS at 45° C. In some embodiments, stringent hybridization conditions should not allow for hybridization of two nucleic acids which differ over a stretch of 20 contiguous nucleotides by more than two bases.

Substitution: As used herein, the term “substitution” refers to the replacement of one or more amino acids or nucleotides by different amino acids or nucleotides, respectively, as compared to the naturally occurring molecule.

Suffering from or Having (a disease): An individual or subject who is “having” or “suffering from” a disease, disorder, and/or condition has been diagnosed with or displays one or more symptoms of the disease, disorder, and/or condition.

Susceptible to: An individual or subject who is “susceptible to” a disease, disorder, and/or condition has not been diagnosed with the disease, disorder, and/or condition. In some embodiments, an individual or subject who is susceptible to a disease, disorder, and/or condition may not exhibit symptoms of the disease, disorder, and/or condition. In some embodiments, an individual or subject who is susceptible to a disease, disorder, and/or condition will develop the disease, disorder, and/or condition. In some embodiments, an individual or subject who is susceptible to a disease, disorder, and/or condition will not develop the disease, disorder, and/or condition.

Solid support: The term “solid support” or “support” means a structure that provides a substrate onto which biomolecules may be bound. For example, a solid support may be an assay well (i.e., such as a microtiter plate), or the solid support may be a location on an array, or a mobile support, such as a bead.

Upstream and downstream: As used herein, the term “upstream” refers to a residue that is N-terminal to a second residue where the molecule is a protein, or 5′ to a second residue where the molecule is a nucleic acid. Also as used herein, the term “downstream” refers to a residue that is C-terminal to a second residue where the molecule is a protein, or 3′ to a second residue where the molecule is a nucleic acid. Protein, polypeptide and peptide sequences disclosed herein are all listed from N-terminal amino acid to C-terminal acid and nucleic acid sequences disclosed herein are all listed from the 5′ end of the molecule to the 3′ end of the molecule.

### Overview

The disclosure herein provides for detection in changes in biomarker expression in a disease and/or syndrome of interest gene that can be used for more accurate diagnosis of disorders relating to the gene and/or syndrome of interest.

In some embodiments, the sample contains nucleic acid. In some embodiments, the testing step comprises nucleic acid sequencing. In some embodiments, the testing step comprises hybridization. In some embodiments, the hybridization is performed using one or more oligonucleotide probes specific for a region in the biomarker of interest. In some embodiments, for detection of mutations associated with changes in biomarker expression, hybridization is performed under conditions sufficiently stringent to disallow a single nucleotide mismatch. In some embodiments, the hybridization is performed with a microarray. In some embodiments, the testing step comprises restriction enzyme digestion. In some embodiments, the testing step comprises PCR amplification. In some embodiments, the PCR amplification is digital PCR amplification. In some embodiments, the testing step comprises primer extension. In some embodiments, the primer extension is single-base primer extension. In some embodiments, the testing step comprises performing a multiplex allele-specific primer extension (ASPE).

In some embodiments, the biomarker is peptide or a protein. In some embodiments, the testing step comprises amino acid sequencing. In some embodiments, the testing step comprises performing an immunoassay using one or more antibodies that specifically recognize the biomarker of interest. In some embodiments, the testing step comprises protease digestion (e.g., trypsin digestion). In some embodiments, the testing step further comprises performing 2D-gel electrophoresis.

In some embodiments, the testing step comprises determining the presence of the one or more biomarkers using mass spectrometry. In some embodiments, the mass spectrometric format is selected from among Matrix-Assisted Laser Desorption/Ionization, Time-of-Flight (MALDI-TOF), Electrospray (ES), IR-MALDI, Ion Cyclotron Resonance (ICR), Fourier Transform, and combinations thereof.

In some embodiments, the sample is a biological sample obtained from cells, tissue (e.g., tissue or liquid biopsy), whole blood, mouthwash, plasma, serum, urine, stool, saliva, cord blood, chorionic villus sample, chorionic villus sample culture, amniotic fluid, amniotic fluid culture, transcervical lavage fluid, cell-free nucleic acid (e.g., DNA or RNA or mRNA or miRNA), or combination thereof. In further embodiments, the sample is obtained from blood or blood products (e.g., plasma or serum) from a pregnant woman and/or fetal DNA. In certain embodiments, the sample is cell-free DNA from plasma, amniotic fluid and the like.

In some embodiments, the testing step comprises determining the amount of the biomarker (e.g., nucleic acid, peptide, protein or other biomolecule such as steroids or cholesterol and the like). In some embodiments, the testing step comprises determining the identity of a nucleotide and/or amino acid at a pre-determined position in the biomarker. In some embodiments, the presence of a mutation in a biomarker is determined by comparing the identity of the nucleotide and/or amino acid at the pre-determined position to a control or non-mutant form of the biomarker.

In embodiments, the method may comprise performing the assay for the biomarker or a mutation in the biomarker (e.g., sequencing) in a plurality of individuals to determine the statistical significance of the association.

In another aspect, the disclosure provides reagents for detecting the biomarker of interest such as, but not limited to a nucleic acid probe that specifically binds to the biomarker (e.g., a particular nucleic acid or a mutation in the nucleic acid sequence), or an array containing one or more probes that specifically bind to the biomarker. In some embodiments, the disclosure provides an antibody that specifically binds to the biomarker. In some embodiments, at least one of the reagents is labeled with a detectable moiety.

In some embodiments, the disclosure provides a kit for comprising one or more of such reagents. In some embodiments, the one or more reagents are provided in a form of microarray. In some embodiments, the kit further comprises reagents for primer extension. In some embodiments, the kit further comprises a negative control that does not contain the biomarker, and/or a control indicative of a healthy individual and/or a positive control having a known amount of the biomarker In some embodiments, at least one of the reagents is labeled with a detectable moiety. In some embodiments, the kit further comprises an instructions on how to determine if an individual has the syndrome or disease of interest based on the biomarker of interest.

In some cases, the amount of the one or more biomarkers may, in certain embodiments, be detected by: (a) detecting the amount of a polypeptide or protein which is regulated by the biomarker; (b) detecting the amount of a polypeptide or protein which regulates the biomarker; or (c) detecting the amount of a metabolite of the biomarker.

In still another aspect, the disclosure herein provides a computer readable medium encoding information corresponding detection of the biomarker.

### Methods and Compositions for Diagnosing Kidney Fibrosis

Embodiments of the present disclosure comprise compositions and methods for diagnosing the presence of, or an increased risk of developing, kidney fibrosis. The methods and compositions of the present disclosure may be used to obtain or provide genetic and/or biochemical information from a subject in order to objectively diagnose the presence or increased risk for that subject, or other subjects to develop kidney fibrosis. The methods and compositions may be embodied in a variety of ways.

In one embodiment, disclosed is a method to detect a biomarker associated with kidney fibrosis in an individual comprising the steps of: obtaining a biological sample from the individual; and measuring the amount of the biomarker, and/or the amount or a mutation in a nucleic acid (e.g. genomic DNA or mRNA) that encodes for, or regulates expression of the biomarker in the biological sample. In an embodiment, the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), NPHS1 nephrin (NPHS1), podicin (NPHS2), nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA). In certain embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or all of the biomarkers are measured. In certain embodiments, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2). In certain embodiments, the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7). In certain embodiments, the biomarker comprises at least one of transforming growth factor beta 1 (TGFB1) or metallopeptidase 2 (MMP2). Additionally and/or alternatively, the method may include measurement of at least one normalization (e.g., housekeeping) gene. In one non-limiting embodiment, the housekeeping gene may be glyceraldehyde 3-phosphate dehydrogenase. Or, other house-keeping genes may be used. Or, measurement of various combinations of these biomarkers may be performed.

Additionally and/or alternatively, other biomarkers may be measured.

As disclosed herein, a variety of methods may be used to measure the biomarkers of interest. In one embodiment, the measuring comprises measuring peptide or polypeptide biomarkers. For example, in one embodiment, the measuring comprises an immunoassay. Or, the measuring may comprise flow cytometry. Or, the measuring may comprise mass spectrometry. Or, as discussed in detail herein, nucleic acid methods may be used.

A variety of biological sample types may be used. In certain embodiments, the sample comprises blood, serum, plasma, a liquid or tissue biopsy, or cell-free nucleic acid. Or, other sample types disclosed herein may be used.

In certain embodiments, the disclosure provides a method of identifying a biomarker associated with kidney fibrosis in an individual. The method may comprise the steps of identifying at least one biomarker having increased or decreased expression in kidney fibrosis as compared to a control individual or population. In an embodiment, the control is a healthy individual with no detected or detectable kidney pathology. In some embodiments, the control is a disease control. Such disease controls may include individuals with kidney disease that is not kidney fibrosis.

In other embodiments, the disclosure provides a method to detect the presence of, or susceptibility to, kidney fibrosis in an individual. The method may comprise the steps of obtaining a biological sample from the individual; measuring the amount of the biomarker, and/or the amount or a mutation in a nucleic acid (e.g. genomic DNA or mRNA) that encodes for, or regulates expression of the biomarker in the sample. In an embodiment, the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), NPHS1 nephrin (NPHS1), podicin (NPHS2), nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA) in the sample, and comparing the measured value for the biomarker with a control value for each of the biomarkers. In certain embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or all of the biomarkers are measured. In certain embodiments, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2). In certain embodiments, the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7). In certain embodiments, the biomarker comprises at least one of transforming growth factor beta 1 (TGFB1) or metallopeptidase 2 (MMP2).

In an embodiment, the control value is determined from a healthy individual or individuals with no detected or detectable lung or cardiovascular pathology. In some embodiments, the control is a disease control. Such disease controls may include individuals with kidney disease that is not kidney fibrosis.

Additionally and/or alternatively, the method may include measurement of at least one normalization (e.g., housekeeping) gene. In one non-limiting embodiment, the housekeeping gene may be glyceraldehyde 3-phosphate dehydrogenase. Or, other house-keeping genes may be used. Or, measurement of various combinations of these biomarkers may be performed.

Additionally and/or alternatively, other biomarkers may be measured.

As disclosed herein, a variety of methods may be used to measure the biomarkers of interest. In one embodiment, the measuring comprises measuring peptide or polypeptide biomarkers. For example, in one embodiment, the measuring comprises an immunoassay. Or, the measuring may comprise flow cytometry. Or, the measuring may comprise flow cytometry. Or, the measuring may comprise mass spectrometry. Or, as discussed in detail herein, nucleic acid methods may be used.

A variety of sample types may be used. In certain embodiments, the biological sample comprises sweat, urine, saliva, a buccal swab, blood, serum, plasma, a liquid or tissue biopsy, or cell-free nucleic acid. Or, other sample types disclosed herein may be used.

Yet other embodiments comprise a composition to detect biomarkers associated with kidney fibrosis in an individual. In certain embodiments, the composition comprises reagents that measure the amount of the biomarker, and/or reagents to measure the amount or detect a mutation in a nucleic acid (e.g. genomic DNA or mRNA) that encodes for the biomarker, or regulates expression of the levels of the biomarker. In an embodiment, the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2), nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA in the sample. In certain embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or all of the biomarkers are measured. In certain embodiments, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2). In certain embodiments, the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7). In certain embodiments, the biomarker comprises at least one of transforming growth factor beta 1 (TGFB1) or metallopeptidase 2 (MMP2).

Additionally and/or alternatively, the composition may include reagents for the measurement of at least one normalization (e.g., housekeeping) gene. In one non-limiting embodiment, the housekeeping gene may be glyceraldehyde 3-phosphate dehydrogenase. Or, other house-keeping genes may be used. Or, measurement of various combinations of these biomarkers may be performed.

For example, as described in detail herein the composition may comprise reagents to measure peptide or polypeptide biomarkers. In one embodiment, the composition comprises reagents to perform an immunoassay. Or, the composition may comprise reagents to perform flow cytometry. Or, the composition may comprise reagents to perform mass spectrometry. Or, as discussed in detail herein, the composition may comprise reagents to determine the presence of a particular sequence and/or expression level of a nucleic acid. For each embodiment, at least some of the reagents (e.g., primers, probes, antibodies, or binding agents) may be labeled with a detectable moiety.

In some embodiments, the composition may be formulated as a kit. Thus, other embodiments include kits that contain at least some of the compositions disclosed herein and/or reagents for performing the methods disclosed herein. In some embodiments, the provided kits comprise a control indicative of a healthy individual, e.g., a nucleic acid and/or protein sample from an individual who does not have the disease and/or syndrome of interest. Or, the kit may comprise a negative control (with no biomarker in a sample) and/or a positive control(s) with a known amount of the biomarker in a sample. For each embodiment, at least some of the reagents (e.g., primers, probes, antibodies, or binding agents) may be labeled with a detectable moiety. Kits may also contain instructions on how to determine if an individual has the disease and/or syndrome of interest, or is at risk of developing the disease and/or syndrome of interest. Such kits may include instructions and/or further information and/or computer-readable media comprising instructions and/or other information for performing the methods.

In certain embodiments, the disclosure provides systems for performing the methods disclosed herein and/or using the compositions described herein.

Peptide, Polypeptide and Protein Assays

In certain embodiments, the biomarker of interest is detected at the protein (or peptide or polypeptide level), that is, a gene product is analyzed. For example, a protein or fragment thereof can be analyzed by amino acid sequencing methods, or immunoassays using one or more antibodies that specifically recognize one or more epitopes present on the biomarker of interest, or in some cases specific to a mutation of interest. Proteins can also be analyzed by protease digestion (e.g., trypsin digestion) and, in some embodiments, the digested protein products can be further analyzed by 2 dimensional-gel electrophoresis.

Antibody Detection

Specific antibodies that bind the biomarker of interest can be employed in any of a variety of methods known in the art. Antibodies against particular epitopes, polypeptides, and/or proteins can be generated using any of a variety of known methods in the art. For example, the epitope, polypeptide, or protein against which an antibody is desired can be produced and injected into an animal, typically a mammal (such as a donkey, mouse, rabbit, horse, chicken, etc.), and antibodies produced by the animal can be collected from the animal. Monoclonal antibodies can also be produced by generating hybridomas that express an antibody of interest with an immortal cell line.

In some embodiments, antibodies and/or other binding agents are labeled with a detectable moiety as described herein.

Antibody detection methods are well known in the art including, but are not limited to, enzyme-linked immunoadsorbent assays (ELISAs) and Western blots. Some such methods are amenable to being performed in an array format.

For example, in some embodiments, the biomarker of interest is detected using a first antibody (or antibody fragment) that specifically recognizes the biomarker. The antibody may be labeled with a detectable moiety (e.g., a chemiluminescent molecule), an enzyme, or a second binding agent (e.g., streptavidin). Or, the first antibody may be detected using a second antibody, as is known in the art.

In certain embodiments, the method may further comprise adding a capture support, the capture support comprising at least one capture support binding agent that recognizes and binds to the biomarker so as to immobilize the biomarker on the capture support. The method may, in certain embodiments, further comprise adding a second binding agent that can specifically recognize and bind to the biomarker and/or at least some of the plurality binding agent molecules on the capture support. In an embodiment, the binding agent that can specifically recognize and bind to the biomarker and/or at least some of the plurality of binding agent molecules on the capture support is a soluble binding agent (e.g., a secondary antibody). The secondary antibody may be labeled (e.g., with an enzyme) such that binding of the biomarker of interest is measured by adding a substrate for the enzyme and quantifying the amount of product formed.

In an embodiment, the capture solid support may be an assay well (i.e., such as a microtiter plate). Or, the capture solid support may be a location on an array, or a mobile support, such as a bead. Or the capture support may be a filter.

In some cases, the biomarker may be allowed to complex with a first binding agent (e.g., primary antibody specific for the biomarker and labeled with detectable moiety) and a second binding agent (e.g., a secondary antibody that recognizes the primary antibody or a second primary antibody), where the second binding agent is complexed to a third binding agent (e.g., biotin) that can then interact with a capture support (e.g., magnetic bead) having a reagent (e.g., streptavidin) that recognizes the third binding agent linked to the capture support. The complex (labeled primary antibody: biomarker: second primary antibody-biotin:streptavidin-bead may then be captured using a magnet (e.g., a magnetic probe) to measure the amount of the complex. Or, alternate versions of such complexes of two or more binding agents such as those known in the art may be employed. For example, in some cases the first binding agent recognizes a second binding agent that is labeled with a detectable moiety such as those described herein, and the second binding agent may recognize (via a ligand present on the or first second binding agent and/or a third binding agent) a capture support.

A variety of binding agents may be used in the methods of the disclosure. For example, the binding agent attached to the capture support, or the second antibody, may be either an antibody or an antibody fragment that recognizes the biomarker. Or, the binding agent may comprise a protein that binds a non-protein target (i.e., such as a protein that specifically binds to a small molecule biomarker, or a receptor that binds to a protein).

In certain embodiments, the solid supports may be treated with a passivating agent. For example, in certain embodiments the biomarker of interest, or a primary antibody or other binding agent that recognizes the biomarker, may be captured on a passivated surface (i.e., a surface that has been treated to reduce non-specific binding). One such passivating agent is BSA. Additionally and/or alternatively, where the binding agent used is an antibody, the solid supports may be coated with protein A, protein G, protein A/G, protein L, or another agent that binds with high affinity to the binding agent (e.g., antibody). These proteins bind the Fc domain of antibodies and thus can orient the binding of antibodies that recognize the protein or proteins of interest.

LC-MS/MS Assays

Liquid chromatography (LC) and/or liquid chromatography-mass spectrometry (e.g., LC-MS/MS) assays may be used for the detection of certain of the biomarkers disclosed herein.

For liquid chromatography (LC), the chromatographic column typically includes a medium (i.e., a packing material) to facilitate separation of chemical moieties (i.e., fractionation). The medium may include minute particles. The particles include a bonded surface that interacts with the various chemical moieties to facilitate separation of the chemical moieties such as the biomarker analytes disclosed herein. One suitable bonded surface is a hydrophobic bonded surface such as an alkyl bonded surface. Alkyl bonded surfaces may include C-4, C-8, or C-18 bonded alkyl groups, preferably C-18 bonded groups. The chromatographic column includes an inlet port for receiving a sample and an outlet port for discharging an effluent that includes the fractionated sample. In the method, the sample (or pre-purified sample) may be applied to the column at the inlet port, eluted with a solvent or solvent mixture, and discharged at the outlet port. Different solvent modes may be selected for eluting different analytes of interest. For example, liquid chromatography may be performed using a gradient mode, an isocratic mode, or a polytyptic (i.e. mixed) mode. In some cases, the LC assay comprises the use of an extraction column for a first purification and an analytical column for a more high resolution purification. The term “analytical column” refers to a chromatography column having sufficient chromatographic plates to effect a separation of the components of a test sample matrix. Preferably, the components eluted from the analytical column are separated in such a way to allow the presence or amount of an analyte(s) of interest to be determined. In some embodiments, the analytical column comprises particles having an average diameter of about 5 μm. In some embodiments, the analytical column is a functionalized silica or polymer-silica hybrid, or a polymeric particle or monolithic silica stationary phase, such as a phenyl-hexyl functionalized analytical column. Analytical columns can be distinguished from “extraction columns,” which typically are used to separate or extract retained materials from non-retained materials to obtained a “purified” sample for further purification or analysis.

Liquid chromatography may, in certain embodiments, comprise high turbulence liquid chromatography or high throughput liquid chromatography (HTLC). See, e.g., Zimmer et al., J. Chromatogr. A 854:23-35 (1999); see also, U.S. Pat. Nos. 5,968,367; 5,919,368; 5,795,469; and 5,772,874. Traditional HPLC analysis relies on column packings in which laminar flow of the sample through the column is the basis for separation of the analyte of interest from the sample. In such columns, separation is a diffusional process. Turbulent flow, such as that provided by HTLC columns and methods, may enhance the rate of mass transfer, improving the separation characteristics provided. In some embodiments, high turbulence liquid chromatography (HTLC), alone or in combination with one or more purification methods, may be used to purify the biomarker of interest prior to mass spectrometry. In such embodiments, samples may be extracted using an HTLC extraction cartridge which captures the analyte, then eluted and chromatographed on a second HTLC column or onto an analytical HPLC column prior to ionization. Because the steps involved in these chromatography procedures can be linked in an automated fashion, the requirement for operator involvement during the purification of the analyte can be minimized. Also, in some embodiments, the use of a high turbulence liquid chromatography sample preparation method can eliminate the need for other sample preparation methods including liquid-liquid extraction. Thus, in some embodiments, the test sample, e.g., a biological fluid, can be disposed, e.g., injected, directly onto a high turbulence liquid chromatography system.

For example, in a typical high turbulence or turbulent liquid chromatography system, the sample may be injected directly onto a narrow (e.g., 0.5 mm to 2 mm internal diameter by 20 to 50 mm long) column packed with large (e.g., >25 micron) particles. When a flow rate (e.g., 3-500 mL per minute) is applied to the column, the relatively narrow width of the column causes an increase in the velocity of the mobile phase. The large particles present in the column can prevent the increased velocity from causing back pressure and promote the formation of vacillating eddies between the particles, thereby creating turbulence within the column.

In high turbulence liquid chromatography, the analyte molecules may bind quickly to the particles and typically do not spread out, or diffuse, along the length of the column. This lessened longitudinal diffusion typically provides better, and more rapid, separation of the analytes of interest from the sample matrix. Further, the turbulence within the column reduces the friction on molecules that typically occurs as they travel past the particles. For example, in traditional HPLC, the molecules traveling closest to the particle move along the column more slowly than those flowing through the center of the path between the particles. This difference in flow rate causes the analyte molecules to spread out along the length of the column. When turbulence is introduced into a column, the friction on the molecules from the particle is negligible, reducing longitudinal diffusion.

The methods and systems of the present disclosure may use mass spectrometry to detect and quantify the biomarker of interest. The terms “mass spectrometry” or “MS” as used herein generally refer to methods of filtering, detecting, and measuring ions based on their mass-to-charge ratio, or “m/z.” In MS techniques, one or more molecules of interest are ionized, and the ions are subsequently introduced into a mass spectrometer where, due to a combination of electric fields, the ions follow a path in space that is dependent upon mass (“m”) and charge (“z”).

In certain embodiments, the mass spectrometer uses a “quadrupole” system. In a “quadrupole” or “quadrupole ion trap” mass spectrometer, ions in an oscillating radio frequency (RF) field experience a force proportional to the direct current (DC) potential applied between electrodes, the amplitude of the RF signal, and m/z. The voltage and amplitude can be selected so that only ions having a particular m/z travel the length of the quadrupole, while all other ions are deflected. Thus, quadrupole instruments can act as both a “mass filter” and as a “mass detector” for the ions injected into the instrument. In certain embodiments, tandem mass spectrometry is used. See, e.g., U.S. Pat. No. 6,107,623, entitled “Methods and Apparatus for Tandem Mass Spectrometry,” which is hereby incorporated by reference in its entirety. In certain embodiments, the selectivity of the MS technique can be enhanced by using “tandem mass spectrometry,” or “MS/MS.” MS/MS methods are useful for the analysis of complex mixtures, especially biological samples, in part because the selectivity of MS/MS can minimize the need for extensive sample clean-up prior to analysis.

In an embodiment, the methods and systems of the present disclosure use a triple quadrupole MS/MS (see e.g., Yost, Enke in Ch. 8 of Tandem Mass Spectrometry, Ed. McLafferty, pub. John Wiley and Sons, 1983). Triple quadrupole MS/MS instruments typically consist of two quadrupole mass filters separated by a fragmentation means. In one embodiment, the instrument may comprise a quadrupole mass filter operated in the RF only mode as an ion containment or transmission device. In an embodiment, the quadropole may further comprise a collision gas at a pressure of between 1 and 10 millitorr. Many other types of “hybrid” tandem mass spectrometers are also known, and can be used in the methods and systems of the present disclosure including various combinations of magnetic sector analyzers and quadrupole filters. These hybrid instruments often comprise high resolution magnetic sector analyzers (i.e., analyzers comprising both magnetic and electrostatic sectors arranged in a double-focusing combination) as either or both of the mass filters. Use of high resolution mass filters may be highly effective in reducing chemical noise to very low levels.

For the methods and systems of the present disclosure, ions can be produced using a variety of methods including, but not limited to, electron ionization, chemical ionization, fast atom bombardment, field desorption, and matrix-assisted laser desorption ionization (“MALDI”), surface enhanced laser desorption ionization (“SELDI”), photon ionization, electrospray ionization, and inductively coupled plasma. A plurality of analytes can be analyzed simultaneously or sequentially by the LC-MS/MS and 2D-LC-MS/MS methods. Exemplary analytes amenable to analysis by the presently disclosed methods include, but are not limited to, vitamins, steroids, peptides, protein, and nucleic acids. One of ordinary skill in the art would recognize after a review of the presently disclosed subject matter that other similar analytes could be analyzed by the methods and systems disclosed herein. Thus, in alternate embodiments, the methods and systems may be used to quantify vitamins, peptide and protein biomarkers, drugs of abuse and therapeutic drugs. For example, optimization of key parameters for each analyte can be performed using a modular method development strategy to provide highly tuned bioanalytical assays. Thus, certain steps may be varied depending upon the analyte being measured as disclosed herein.

Also, embodiments of the methods and systems of the present disclosure may provide greater sensitivity than the sensitivities previously attainable for many of the analytes being measured. For example, through using this optimization procedure, a lower limit of quantitation (LOQ) of about 0.05 milligram per deciliter (mg/dL), or less than 0.1 mg/dL, or less than 1 mg/dL, or less than 5 mg/dL is attained. The levels of detection may allow for the analysis of sample volumes ranging from 0.5 mL to greater than 1 mL.

Nucleic Acid Assays

In certain embodiments, the biomarkers disclosed herein are detected at the nucleic acid level. In one embodiment, the disclosure comprises methods for diagnosing the presence or an increased risk of developing the syndrome or disease of interest (e.g., kidney fibrosis) in a subject. The method may comprise the steps of obtaining a nucleic acid from a tissue or body fluid sample from a subject and conducting an assay to measure the amount of a nucleic acid (e.g., DNA or RNA or mRNA) biomarker.

Additionally and/or alternatively, the method may comprise the steps of obtaining a nucleic acid from a tissue or body fluid sample from a subject and conducting an assay to identify whether there is a variant sequence (i.e., a mutation) in the subject's nucleic acid. In certain embodiments, the method may comprise comparing the variant to known variants associated with the syndrome or disease of interest and determining whether the variant is a variant that has been previously identified as being associated with the syndrome or disease of interest. Or, the method may comprise identifying the variant as a new, previously uncharacterized variant. If the variant is a new variant, the method may further comprise performing an analysis to determine whether the mutation is expected to be deleterious to expression of the gene and/or the function of the protein encoded by the gene. The method may further comprise using the variant profile (i.e., the compilation of mutations identified in the subject) to diagnose the presence of the syndrome or disease of interest or an increased risk of developing the syndrome or disease of interest.

Nucleic acid analyses can be performed on genomic DNA (including cell free DNA), messenger RNAs (including cell free RNA), and/or cDNA made from RNA. Also, in various embodiments, the nucleic acid comprises a gene, an RNA, an exon, an intron, a gene regulatory element, an expressed RNA, an siRNA, or an epigenetic element. Also, regulatory elements, including splice sites, transcription factor binding, A-I editing sites, microRNA binding sites, and functional RNA structure sites may be evaluated for mutations (i.e., variants). Thus, for each of the methods and compositions of the disclosure, the variant may comprise a nucleic acid sequence that encompasses at least one of the following: (1) A-to-I editing sites ; (2) splice sites; (3) conserved functional RNA structures; (4) validated transcription factor binding sites (TFBS); (5) microRNA (miRNA) binding sites; (6) polyadenylation sites; (7) known regulatory elements; (8) miRNA genes; (9) small nucleolar RNA genes encoded in the ROIs; and/or (10) ultra-conserved elements across placental mammals.

In many embodiments, nucleic acids are extracted from a biological sample. In some embodiments, nucleic acids are analyzed without having been amplified. In some embodiments, nucleic acids are amplified using techniques known in the art (such as polymerase chain reaction (PCR)), quantitative PCR and/or real-time PCR (to determine nucleic acid amount), and amplified nucleic acids are used in subsequent analyses. Multiplex PCR, in which several amplicons (e.g., from different genomic regions) are amplified at once using multiple sets of primer pairs, may be employed. For example, nucleic acid can be analyzed by sequencing, hybridization, PCR amplification, restriction enzyme digestion, primer extension such as single-base primer extension or multiplex allele-specific primer extension (ASPE), or DNA sequencing. In some embodiments, nucleic acids are amplified in a manner such that the amplification product for a wild-type allele differs in size from that of a mutant allele. Thus, presence or absence of a particular mutant allele can be determined by detecting size differences in the amplification products, e.g., on an electrophoretic gel. For example, deletions or insertions of gene regions may be particularly amenable to using size-based approaches.

Certain exemplary nucleic acid analysis methods are described in detail below.

Allele-Specific Amplification

In some embodiments, for example, where the biomarker for the disease and/or syndrome of interest is a mutation, a biomarker is detected using an allele-specific amplification assay. This approach is variously referred to as PCR amplification of specific allele (PASA) (Sarkar, et al., 1990 Anal. Biochem. 186:64-68), allele-specific amplification (ASA) (Okayama, et al., 1989 J. Lab. Clin. Med. 114:105-113), allele-specific PCR (ASPCR) (Wu, et al. 1989 Proc. Natl. Acad. Sci. USA. 86:2757-2760), and amplification-refractory mutation system (ARMS) (Newton, et al., 1989 Nucleic Acids Res. 17:2503-2516). The entire contents of each of these references is incorporated herein. This method is applicable for single base substitutions as well as micro deletions/insertions.

For example, for PCR-based amplification methods, amplification primers may be designed such that they can distinguish between different alleles (e.g., between a wild-type allele and a mutant allele). Thus, the presence or absence of amplification product can be used to determine whether a gene mutation is present in a given nucleic acid sample. In some embodiments, allele specific primers can be designed such that the presence of amplification product is indicative of the gene mutation. In some embodiments, allele specific primers can be designed such that the absence of amplification product is indicative of the gene mutation.

In some embodiments, two complementary reactions are used. One reaction employs a primer specific for the wild type allele (“wild-type-specific reaction”) and the other reaction employs a primer for the mutant allele (“mutant-specific reaction”). The two reactions may employ a common second primer. PCR primers specific for a particular allele (e.g., the wild-type allele or mutant allele) generally perfectly match one allelic variant of the target, but are mismatched to other allelic variant (e.g., the mutant allele or wild-type allele). The mismatch may be located at/near the 3′ end of the primer, leading to preferential amplification of the perfectly matched allele. Whether an amplification product can be detected from one or in both reactions indicates the absence or presence of the mutant allele. Detection of an amplification product only from the wild-type-specific reaction indicates presence of the wild-type allele only (e.g., homozygosity of the wild-type allele). Detection of an amplification product in the mutant-specific reaction only indicates presence of the mutant allele only (e.g. homozygosity of the mutant allele). Detection of amplification products from both reactions indicate (e.g., a heterozygote). As used herein, this approach will be referred to as “allele specific amplification (ASA).”

Allele-specific amplification can also be used to detect duplications, insertions, or inversions by using a primer that hybridizes partially across the junction. The extent of junction overlap can be varied to allow specific amplification.

Amplification products can be examined by methods known in the art, including by visualizing (e.g., with one or more dyes) bands of nucleic acids that have been migrated (e.g., by electrophoresis) through a gel to separate nucleic acids by size.

Allele-Specific Primer Extension

In some embodiments, an allele-specific primer extension (ASPE) approach is used to detect a gene mutations. ASPE employs allele-specific primers that can distinguish between alleles (e.g., between a mutant allele and a wild-type allele) in an extension reaction such that an extension product is obtained only in the presence of a particular allele (e.g., mutant allele or wild-type allele). Extension products may be detectable or made detectable, e.g., by employing a labeled deoxynucleotide in the extension reaction. Any of a variety of detectable moieties (i.e., labels) are compatible for use in these methods, including, but not limited to, radioactive labels, fluorescent labels, chemiluminescent labels, enzymatic labels, etc. In some embodiments, a nucleotide is labeled with an entity that can then be bound (directly or indirectly) by a detectable label, e.g., a biotin molecule that can be bound by streptavidin-conjugated fluorescent dyes. In some embodiments, reactions are done in multiplex, e.g., using many allele-specific primers in the same extension reaction.

In some embodiments, extension products are hybridized to a solid or semi-solid support, such as beads, matrix, gel, among others. For example, the extension products may be tagged with a particular nucleic acid sequence (e.g., included as part of the allele-specific primer) and the solid support may be attached to an “anti-tag” (e.g., a nucleic acid sequence complementary to the tag in the extension product). Extension products can be captured and detected on the solid support. For example, beads may be sorted and detected. One such system that can be employed in this manner is the LUM1NEX™ MAP system, which can be adapted for cystic fibrosis mutation detection by TM Bioscience and is sold commercially as a universal bead array (TAG-IT™)

Single Nucleotide Primer Extension

In some embodiments, a single nucleotide primer extension (SNuPE) assay is used, in which the primer is designed to be extended by only one nucleotide. In such methods, the identity of the nucleotide just downstream of the 3′ end of the primer is known and differs in the mutant allele as compared to the wild-type allele. SNuPE can be performed using an extension reaction in which the only one particular kind of deoxynucleotide is labeled (e.g., labeled dATP, labeled dCTP, labeled dGTP, or labeled dTTP). Thus, the presence of a detectable extension product can be used as an indication of the identity of the nucleotide at the position of interest (e.g., the position just downstream of the 3′ end of the primer), and thus as an indication of the presence or absence of a mutation at that position. SNuPE can be performed as described in U.S. Pat. No. 5,888,819; U.S. Pat. No. 5,846,710; U.S. Pat. No. 6,280,947; U.S. Pat. No. 6,482,595; U.S. Pat. No. 6,503,718; U.S. Pat. No. 6,919,174; Piggee, C. et al. Journal of Chromatography A 781 (1997), p. 367-375 (“Capillary Electrophoresis for the Detection of Known Point Mutations by Single-Nucleotide Primer Extension and Laser-Induced Fluorescence Detection”); Hoogendoorn, B. et al., Human Genetics (1999) 104:89-93, (“Genotyping Single Nucleotide Polymorphism by Primer Extension and High Performance Liquid Chromatography”).

In some embodiments, primer extension can be combined with mass spectrometry for accurate and fast detection of the presence or absence of a mutation. See, U.S. Pat. No. 5,885,775 to Haff et al. (analysis of single nucleotide polymorphism analysis by mass spectrometry); U.S. Pat. No. 7,501,251 to Koster (DNA diagnosis based on mass spectrometry). Suitable mass spectrometric format includes, but is not limited to, Matrix-Assisted Laser Desorption/Ionization, Time-of-Flight (MALDI-TOF), Electrospray (ES), IR-MALDI, Ion Cyclotron Resonance (ICR), Fourier Transform, and combinations thereof.

Oligonucleotide Ligation Assay

In some embodiments, an oligonucleotide ligation assay (“OLA” or “OL”) is used. OLA employs two oligonucleotides that are designed to be capable of hybridizing to abutting sequences of a single strand of a target molecules. Typically, one of the oligonucleotides is biotinylated, and the other is detectably labeled, e.g., with a streptavidin-conjugated fluorescent moiety. If the precise complementary sequence is found in a target molecule, the oligonucleotides will hybridize such that their termini abut, and create a ligation substrate that can be captured and detected. See e.g., Nickerson et al. (1990) Proc. Natl. Acad. Sci. U.S.A. 87:8923-8927, Landegren, U. et al. (1988) Science 241:1077-1080 and U.S. Pat. No. 4,998,617.

Hybridization Approach

In some embodiments, the presence of a nucleic acid is detected and/or quantified by hybridization to a probe that include at least a portion of the sequence.

In some embodiments, nucleic acids are analyzed by hybridization using one or more oligonucleotide probes specific for the biomarker of interest and under conditions sufficiently stringent to disallow a single nucleotide mismatch. In certain embodiments, suitable nucleic acid probes can distinguish between a normal gene and a mutant gene. Thus, for example, one of ordinary skill in the art could use probes of the invention to determine whether an individual is homozygous or heterozygous for a particular allele.

Nucleic acid hybridization techniques are well known in the art. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

In some embodiments, probe molecules that hybridize biomarker nucleic acid sequences can be used for detecting such sequences in the amplified product by solution phase or, more preferably, solid phase hybridization. Solid phase hybridization can be achieved, for example, by attaching probes to a microchip.

Nucleic acid probes may comprise ribonucleic acids and/or deoxyribonucleic acids. In some embodiments, provided nucleic acid probes are oligonucleotides (i.e., “oligonucleotide probes”). Generally, oligonucleotide probes are long enough to bind specifically to a homologous region of the gene of interest, but short enough such that a difference of one nucleotide between the probe and the nucleic acid sample being tested disrupts hybridization. Typically, the sizes of oligonucleotide probes vary from approximately 10 to 100 nucleotides. In some embodiments, oligonucleotide probes vary from 15 to 90, 15 to 80, 15 to 70, 15 to 60, 15 to 50, 15 to 40, 15 to 35, 15 to 30, 18 to 30, or 18 to 26 nucleotides in length. As appreciated by those of ordinary skill in the art, the optimal length of an oligonucleotide probe may depend on the particular methods and/or conditions in which the oligonucleotide probe may be employed.

In some embodiments, nucleic acid probes are useful as primers, e.g., for nucleic acid amplification and/or extension reactions. For example, in certain embodiments, the gene sequence being evaluated for a variant comprises the exon sequences. In certain embodiments, the exon sequence and additional flanking sequence (e.g., about 5, 10, 15, 20, 25, 30, 35, 40, 45, 50, 55 or more nucleotides of UTR and/or intron sequence) is analyzed in the assay. Or, intron sequences or other non-coding regions may be evaluated for potentially deleterious mutations. Or, portions of these sequences may be used. Such variant gene sequences may include sequences having at least one of the mutations as described herein.

Other embodiments of the disclosure provide isolated gene sequences containing mutations that relate to the syndrome and/or disease of interest. Such gene sequences may be used to objectively diagnose the presence or increased risk for a subject to develop kidney fibrosis. In certain embodiments, the isolated nucleic acid may contain a non-variant sequence or a variant sequence of any one or combination thereof. For example, in certain embodiments, the gene sequence comprises the exon sequences. In certain embodiments, the exon sequence and additional flanking sequence (e.g., about 5, 10, 15, 20, 25, 30, 35, 40, 45, 50, 55 or more nucleotides of UTR and/or intron sequence) is analyzed in the assay. Or, intron sequences or other non-coding regions may be used. Or, portions of these sequences may be used. In certain embodiments, the gene sequence comprises an exon sequence from at least one of the biomarker genes disclosed herein.

In some embodiments, nucleic acid probes are labeled with a detectable moiety as described herein.

Quantitative and Real-Time PCR

Quantitative PCR are known in the art and may be used to quantify of the amount of a target biomarker nucleic acid sequence that is present at the start at the PCR reaction. The principles of real-time PCR are generally described, for example, in Held et al. “Real Time Quantitative PCR” Genome Research 6:986-994 (1996). Generally, real-time PCR measures a signal at each amplification cycle. Some real-time PCR techniques rely on fluorophores that emit a signal at the completion of every multiplication cycle. Examples of such fluorophores are fluorescence dyes that emit fluorescence at a defined wavelength upon binding to double-stranded DNA, such as SYBR green. An increase in double-stranded DNA during each amplification cycle thus leads to an increase in fluorescence intensity due to accumulation of PCR product. Another example of fluorophores used for detection in real-time PCR are sequence-specific fluorescent reporter probes, described elsewhere in this document. The examples of such probes are TaqMan® probes. The use of sequence-specific reporter probe provides for detection of a target sequence with high specificity, and enables quantification even in the presence of non-specific DNA amplification. Fluorescent probes can also be used in multiplex assays—for detection of several genes in the same reaction—based on specific probes with different-colored labels. For example, a multiplex assay can use several sequence-specific probes, labeled with a variety of fluorophores, including, but not limited to, FAM, JA270, CY5.5, and HEX, in the same PCR reaction mixture.

Real-time PCR relies on detection of a measurable parameter, such as fluorescence, during the course of the PCR reaction. The amount of the measurable parameter is proportional to the amount of the PCR product, which allows one to observe the increase of the PCR product “in real time.” Some real-time PCR methods allow for quantification of the input DNA template based on the observable progress of the PCR reaction. The analysis and processing of the data is discussed below. A “growth curve” or “amplification curve” in the context of a nucleic acid amplification assay is a graph of a function, where an independent variable is the number of amplification cycles and a dependent variable is an amplification-dependent measurable parameter measured at each cycle of amplification, such as fluorescence emitted by a fluorophore. As discussed above, the amount of amplified target nucleic acid can be detected using a fluorophore-labeled probe. Typically, the amplification-dependent measurable parameter is the amount of fluorescence emitted by the probe upon hybridization, or upon the hydrolysis of the probe by the nuclease activity of the nucleic acid polymerase. The increase in fluorescence emission is measured in real time and is directly related to the increase in target nucleic acid amplification (such as influenza nucleic acid amplification). In some examples, the change in fluorescence (dRₙ) is calculated using the equation dRₙ=Rₙ₊−Rₙ—, with Rₙ₊ being the fluorescence emission of the product at each time point and Rₙ− being the fluorescence emission of the baseline. The dRₙ values are plotted against cycle number, resulting in amplification plots. In a typical polymerase chain reaction, a growth curve contains a segment of exponential growth followed by a plateau, resulting in a sigmoidal-shaped amplification plot when using a linear scale. A growth curve is characterized by a “cross point” value or “Cₚ” value, which can be also termed “threshold value” or “cycle threshold” (Cₜ), which is a number of cycles where a predetermined magnitude of the measurable parameter is achieved. For example, when a fluorophore-labeled probe is employed, the threshold value (Cₜ) is the PCR cycle number at which the fluorescence emission (dRₙ) exceeds a chosen threshold, which is typically 10 times the standard deviation of the baseline (this threshold level can, however, be changed if desired). A lower Cₜ value represents more rapid completion of amplification, while the higher Cₜ value represents slower completion of amplification. Where efficiency of amplification is similar, the lower Cₜ value is reflective of a higher starting amount of the target nucleic acid, while the higher Cₜ value is reflective of a lower starting amount of the target nucleic acid. Where a control nucleic acid of known concentration is used to generate a “standard curve,” or a set of “control” Cₜ values at various known concentrations of a control nucleic acid, it becomes possible to determine the absolute amount of the target nucleic acid in the sample by comparing Cₜ values of the target and control nucleic acids.

Arrays

A variety of the methods mentioned herein may be adapted for use with arrays that allow sets of biomarkers to be analyzed and/or detected in a single experiment. For example, multiple biomarkers (or portions thereof) and/or multiple mutations that comprise biomarkers can be analyzed at the same time. In particular, methods that involve use of nucleic acid reagents (e.g., probes, primers, oligonucleotides, etc.) are particularly amenable for adaptation to an array-based platform (e.g., microarray). In some embodiments, an array containing one or more probes specific for detecting mutations in the biomarker of interest.

DNA Sequencing

In certain embodiments, diagnosis of the biomarker of interest is carried out by detecting variation in the sequence, genomic location or arrangement, and/or genomic copy number of a nucleic acid or a panel of nucleic acids by nucleic acid sequencing.

In some embodiments, the method may comprise obtaining a nucleic acid from a tissue or body fluid sample from a subject and sequencing at least a portion of a nucleic acid in order to obtain a sample nucleic acid sequence for at least one gene. In certain embodiments, the method may comprise comparing the variant to known variants associated with kidney fibrosis and determining whether the variant is a variant that has been previously identified as being associated with kidney fibrosis. Or, the method may comprise identifying the variant as a new, previously uncharacterized variant. If the variant is a new variant, or in some cases for previously characterized (i.e., identified) variants, the method may further comprise performing an analysis to determine whether the mutation is expected to be deleterious to expression of the gene and/or the function of the protein encoded by the gene. The method may further comprise using the variant profile (i.e., a compilation of variants identified in the subject) to diagnose the presence of kidney fibrosis or an increased risk of developing kidney fibrosis.

For example, in certain embodiments, next generation (massively-parallel sequencing) may be used. Or, Sanger sequencing may be used. Or, a combination of next-generation (massively-parallel sequencing) and Sanger sequencing may be used. Additionally and/or alternatively, the sequencing comprises at least one of single-molecule sequencing-by-synthesis. Thus, in certain embodiments, a plurality of DNA samples are analyzed in a pool to identify samples that show a variation. Additionally or alternatively, in certain embodiments, a plurality of DNA samples are analyzed in a plurality of pools to identify an individual sample that shows the same variation in at least two pools.

One conventional method to perform sequencing is by chain termination and gel separation, as described by Sanger et al., 1977, Proc Natl Acad Sci U S A, 74:5463-67. Another conventional sequencing method involves chemical degradation of nucleic acid fragments. See, Maxam et al., 1977, Proc. Natl. Acad. Sci., 74:560-564. Also, methods have been developed based upon sequencing by hybridization. See, e.g., Harris et al., U.S. Patent Application Publication No. 20090156412.

In other embodiments, sequencing of the nucleic acid is accomplished by massively parallel sequencing (also known as “next generation sequencing”) of single-molecules or groups of largely identical molecules derived from single molecules by amplification through a method such as PCR. Massively parallel sequencing is shown for example in Lapidus et al., U.S. Pat. No. 7,169,560, Quake et al. U.S. Pat. No. 6,818,395, Harris U.S. Pat. No. 7,282,337 and Braslaysky, et al., PNAS (USA), 100: 3960-3964 (2003), the contents of each of which are incorporated by reference herein.

In next generation sequencing, PCR or whole genome amplification can be performed on the nucleic acid in order to obtain a sufficient amount of nucleic acid for analysis. In some forms of next generation sequencing, no amplification is required because the method is capable of evaluating DNA sequences from unamplified DNA. Once determined, the sequence and/or genomic arrangement and/or genomic copy number of the nucleic acid from the test sample is compared to a standard reference derived from one or more individuals not known to suffer from kidney fibrosis at the time their sample was taken. All differences between the sequence and/or genomic arrangement and/or genomic arrangement and/or copy number of the nucleic acid from the test sample and the standard reference are considered variants.

In next generation (massively parallel sequencing), all regions of interest are sequenced together, and the origin of each sequence read is determined by comparison (alignment) to a reference sequence. The regions of interest can be enriched together in one reaction, or they can be enriched separately and then combined before sequencing. In certain embodiments, the DNA sequences derived from coding exons of genes included in the assay are enriched by bulk hybridization of randomly fragmented genomic DNA to specific RNA probes. The same adapter sequences are attached to the ends of all fragments, allowing enrichment of all hybridization-captured fragments by PCR with one primer pair in one reaction. Regions that are less efficiently captured by hybridization are amplified by PCR with specific primers. In addition, PCR with specific primers is may be used to amplify exons for which similar sequences (“pseudo exons”) exist elsewhere in the genome.

In certain embodiments where massively parallel sequencing is used, PCR products are concatenated to form long stretches of DNA, which are sheared into short fragments (e.g., by acoustic energy). This step ensures that the fragment ends are distributed throughout the regions of interest. Subsequently, a stretch of dA nucleotides is added to the 3′ end of each fragment, which allows the fragments to bind to a planar surface coated with oligo(dT) primers (the “flow cell”). Each fragment may then be sequenced by extending the oligo(dT) primer with fluorescently-labeled nucleotides. During each sequencing cycle, only one type of nucleotide (A, G, T, or C) is added, and only one nucleotide is allowed to be incorporated through use of chain terminating nucleotides. For example, during the 1st sequencing cycle, a fluorescently labeled dCTP could be added. This nucleotide will only be incorporated into those growing complementary DNA strands that need a C as the next nucleotide. After each sequencing cycle, an image of the flow cell is taken to determine which fragment was extended. DNA strands that have incorporated a C will emit light, while DNA strands that have not incorporated a C will appear dark. Chain termination is reversed to make the growing DNA strands extendible again, and the process is repeated for a total of 120 cycles.

The images are converted into strings of bases, commonly referred to as “reads,” which recapitulate the 3′ terminal 25 to 60 bases of each fragment. The reads are then compared to the reference sequence for the DNA that was analyzed. Since any given string of 25 bases typically only occurs once in the human genome, most reads can be “aligned” to one specific place in the human genome. Finally, a consensus sequence of each genomic region may be built from the available reads and compared to the exact sequence of the reference at that position. Any differences between the consensus sequence and the reference are called as sequence variants.

Detectable Moieties

In certain embodiments, certain molecules (e.g., nucleic acid probes, antibodies, etc.) used in accordance with and/or provided by the invention comprise one or more detectable entities or moieties, i.e., such molecules are “labeled” with such entities or moieties.

Any of a wide variety of detectable agents can be used in the practice of the disclosure. Suitable detectable agents include, but are not limited to: various ligands, radionuclides; fluorescent dyes; chemiluminescent agents (such as, for example, acridinum esters, stabilized dioxetanes, and the like); bioluminescent agents; spectrally resolvable inorganic fluorescent semiconductors nanocrystals (i.e., quantum dots); microparticles; metal nanoparticles (e.g., gold, silver, copper, platinum, etc.); nanoclusters; paramagnetic metal ions; enzymes; colorimetric labels (such as, for example, dyes, colloidal gold, and the like); biotin; dioxigenin; haptens; and proteins for which antisera or monoclonal antibodies are available.

In some embodiments, the detectable moiety is biotin. Biotin can be bound to avidins (such as streptavidin), which are typically conjugated (directly or indirectly) to other moieties (e.g., fluorescent moieties) that are detectable themselves.

Below are described some non-limiting examples of some detectable moieties that may be used.

Fluorescent Dyes

In certain embodiments, a detectable moiety is a fluorescent dye. Numerous known fluorescent dyes of a wide variety of chemical structures and physical characteristics are suitable for use in the practice of the disclosure. A fluorescent detectable moiety can be stimulated by a laser with the emitted light captured by a detector. The detector can be a charge-coupled device (CCD) or a confocal microscope, which records its intensity.

Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), hexachloro-fluorescein (HEX), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Q-DOTS. Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, CY-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY R, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. For more examples of suitable fluorescent dyes and methods for coupling fluorescent dyes to other chemical entities such as proteins and peptides, see, for example, “The Handbook of Fluorescent Probes and Research Products”, 9th Ed., Molecular Probes, Inc., Eugene, Oreg. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable moiety may include more than one chemical entity such as in fluorescent resonance energy transfer (FRET). Resonance transfer results an overall enhancement of the emission intensity. For instance, see Ju et. al. (1995) Proc. Nat'l Acad. Sci. (USA) 92:4347, the entire contents of which are herein incorporated by reference. To achieve resonance energy transfer, the first fluorescent molecule (the “donor” fluor) absorbs light and transfers it through the resonance of excited electrons to the second fluorescent molecule (the “acceptor” fluor). In one approach, both the donor and acceptor dyes can be linked together and attached to the oligo primer. Methods to link donor and acceptor dyes to a nucleic acid have been described, for example, in U.S. Pat. No. 5,945,526 to Lee et al. Donor/acceptor pairs of dyes that can be used include, for example, fluorescein/tetramethylrohdamine, IAEDANS/fluroescein, EDANS/DABCYL, fluorescein/fluorescein, BODIPY FL/BODIPY FL, and Fluorescein/QSY 7 dye. See, e.g., U.S. Pat. No. 5,945,526 to Lee et al. Many of these dyes also are commercially available, for instance, from Molecular Probes Inc. (Eugene, Oreg.). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

Enzymes

In certain embodiments, a detectable moiety is an enzyme. Examples of suitable enzymes include, but are not limited to, those used in an ELISA, e.g., horseradish peroxidase, beta-galactosidase, luciferase, alkaline phosphatase, etc. Other examples include beta-glucuronidase, beta-D-glucosidase, urease, glucose oxidase, etc. An enzyme may be conjugated to a molecule using a linker group such as a carbodiimide, a diisocyanate, a glutaraldehyde, and the like.

Radioactive Isotopes

In certain embodiments, a detectable moiety is a radioactive isotope. For example, a molecule may be isotopically-labeled (i.e., may contain one or more atoms that have been replaced by an atom having an atomic mass or mass number different from the atomic mass or mass number usually found in nature) or an isotope may be attached to the molecule. Non-limiting examples of isotopes that can be incorporated into molecules include isotopes of hydrogen, carbon, fluorine, phosphorous, copper, gallium, yttrium, technetium, indium, iodine, rhenium, thallium, bismuth, astatine, samarium, and lutetium (i.e., 3H, 13C, 14C, 18F, 19F, 32P, 35S, 64Cu, 67Cu, 67Ga, 90Y, 99mTc, 111In, 125I, 123I, 129I, 131I, 135I, 186Re, 187Re, 201Tl, 212Bi, 213Bi, 211At, 153Sm, 177Lu).

In some embodiments, signal amplification is achieved using labeled dendrimers as the detectable moiety (see, e.g., Physiol Genomics 3:93-99, 2000), the entire contents of which are herein incorporated by reference in their entirety. Fluorescently labeled dendrimers are available from Genisphere (Montvale, N.J.). These may be chemically conjugated to the oligonucleotide primers by methods known in the art.

Systems

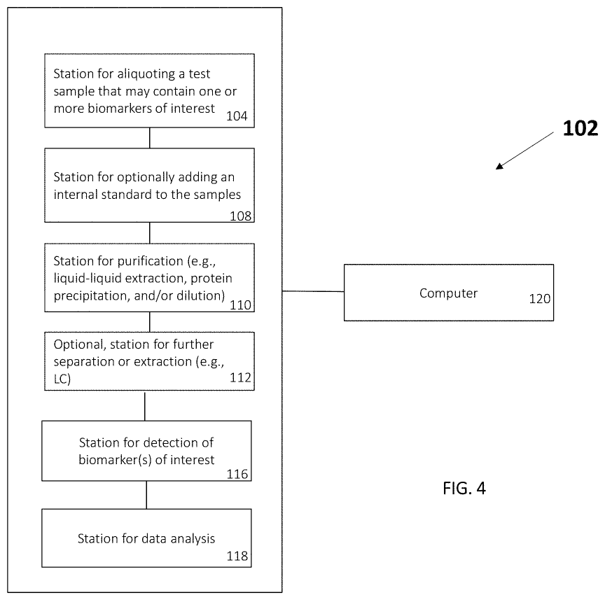

In certain embodiments, the disclosure provides systems for performing the methods disclosed herein and/or using the compositions described herein. In certain embodiments, the system may comprise a kit. Or, the system may comprise computerized instructions and/or reagents for performing the methods disclosed herein. For example, a system of the invention is shown in FIG. 4.

For example, in some embodiments, the system may comprise: a station for providing a sample (e.g., a biological sample) believed to contain at least one biomarker of interest; optionally, a station for separating the at least one biomarker of interest from other components in the sample; a station for measuring the amount of the biomarker of interest; and a station to analyze the results to determine the presence or amount of the one or more biomarkers in the sample. Also in certain embodiments, at least one of the stations is automated and/or controlled by a computer.

In an embodiment, the biomarkers comprise the biomarker, and/or the amount or a mutation in a nucleic acid (e.g. genomic DNA or mRNA) that encodes for, or regulates expression of the biomarker. The biomarker may comprise at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2), nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA). In certain embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or all of the biomarkers are measured. In certain embodiments, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2). In certain embodiments, the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7). In certain embodiments, the biomarker comprises at least one of transforming growth factor beta 1 (TGFB1) or metallopeptidase 2 (MMP2).

Additionally and/or alternatively, the system may include measurement of at least one normalization (e.g., housekeeping) gene. In one non-limiting embodiment, the housekeeping gene may be glyceraldehyde 3-phosphate dehydrogenase. Or, other housekeeping genes may be used. Or, measurement of various combinations of these biomarkers may be performed.

The system may comprise a station for at least partially purifying the biomarker. In an embodiment, the station for purification comprises a station for protein precipitation. The station for purifying the biomarker may comprise components for extracting the biomarker of interest from the test sample and/or diluting the sample. In an embodiment, the station for extraction comprises a station for liquid-liquid extraction. The station for liquid-liquid extraction may comprise equipment and reagents for addition of solvents to the sample and removal of waste fractions. In some cases a isotopically-labeled internal standard is used to standardize losses of the biomarker that may occur during the procedures. Thus, the station for purification (e.g., liquid-liquid extraction) may comprise a hood or other safety features required for working with solvents.

In certain embodiments, the methods and systems of the present disclosure may comprise liquid chromatography or multiple liquid chromatography steps. In certain embodiments, a two-dimensional liquid chromatography (LC) procedure is used. For example, in one embodiment, the method and systems of the present disclosure may comprise transferring the biomarker of interest from the LC extraction column to an analytical column. In one embodiment, the transferring of the at least one biomarker of interest from the extraction column to an analytical column is done by a heart-cutting technique. In another embodiment, the biomarker of interest is transferred from the extraction column to an analytical column by a chromato-focusing technique. Alternatively, the biomarker of interest is transferred from the extraction column to an analytical column by a column switching technique. These transfer steps may be done manually, or may be part of an on-line system. Optionally, an extraction column may not be used in the methods and systems described herein.

Various columns comprising stationary phases and mobile phases that may be used for extraction or analytical liquid chromatography are described herein. The column used for optional extraction liquid chromatography may be varied depending on the biomarker of interest. The column used for analytical liquid chromatography may be varied depending on the biomarker of interest and/or the column that was used for the extraction liquid chromatography step. For example, in certain embodiments, the analytical column comprises particles having an average diameter of about 3 p.m. In some embodiments, the analytical column is a functionalized silica or polymer-silica hybrid, or a polymeric particle or monolithic silica stationary phase, such as a phenyl-hexyl functionalized analytical column.

After separation from other components, the biomarker may be analyzed by any one of the techniques described herein (e.g., immunodetection, LC-MS/MS, measurement of RNA, DNA and/or other techniques disclosed herein).

FIG. 4 provides a drawing of an embodiment of a system (102) of the disclosure. As shown in FIG. 4, the system may comprise a station for aliquoting a sample (104) that may comprise a biomarker of interest into sampling containers. In one embodiment, the sample is aliquoted into a container or containers to facilitate liquid-liquid extraction or sample dilution. The station for aliquoting may comprise receptacles to discard the portion of the biological sample that is not used in the analysis.

The system may further comprise a station for adding an internal standard to the sample (108). In an embodiment, the internal standard comprises the biomarker of interest labeled with a non-natural isotope. Thus, the station for adding an internal standard may comprise safety features to facilitate adding an isotopically labeled internal standard solutions to the sample. The system may also, in some embodiments, comprise a station for purification as e.g., by liquid-liquid extraction, protein precipitation and/or dilution of the sample (110).

The system may also comprise a station for further purification as e.g., by liquid chromatography (LC) of the sample. As described herein, in an embodiment, the station for liquid chromatography may comprise an extraction liquid chromatography or HPLC column and/or an analytical LC or HPLC column (112) and/or an HTLC column or other columns described herein. The station for liquid chromatography may comprise a column(s) comprising the stationary phase, as well as containers or receptacles comprising solvents that are used as the mobile phase. In an embodiment, the mobile phase comprises a gradient of acetonitrile, ammonium formate, and water, or other miscible solvents with aqueous volatile buffer solutions. Thus, in one embodiment, the station may comprise the appropriate lines and valves to adjust the amounts of individual solvents being applied to the column or columns. Also, the station may comprise a means to remove and discard those fractions from the LC that do not comprise the biomarker of interest. In an embodiment, the fractions that do not contain the biomarker of interest are continuously removed from the column and sent to a waste receptacle for decontamination and to be discarded.

A variety of extraction LC systems may be used. For example, in the embodiment where the system is being used to measure ascorbic acid, an extraction column with an analytical column, with mobile phases comprising a gradient of acetonitrile and water are used. The system may also comprise an analytical LC column, generally included after the extraction column (not shown in FIG. 4). The analytical column may facilitate further purification and concentration of the biomarker of interest as may be required for further characterization and quantification.

Also, the system may comprise a station for characterization and quantification of the biomarker of interest (116). In one embodiment, the system may comprise a station for mass spectrometry (MS) and or tandem mass spectrometry (MS/MS) of the biomarker. Or, other measurement techniques may be used such as any of the measurement techniques described herein. Also, the station for characterization and quantification may comprise a computer and software for analysis of the results (118). In an embodiment, the analysis comprises both identification and quantification of the biomarker of interest.

In some embodiments, one or more of the purification or separation steps can be performed “on-line.” As used herein, the term “on-line” refers to purification or separation steps that are performed in such a way that the test sample is disposed, e.g., injected, into a system in which the various components of the system are operationally connected and, in some embodiments, in fluid communication with one another. The on-line system may comprise an autosampler for removing aliquots of the sample from one container and transferring such aliquots into another container.

In some embodiments, the on-line purification or separation method can be automated. In such embodiments, the steps can be performed without the need for operator intervention once the process is set-up and initiated. For example, in one embodiment, the system, or portions of the system may be controlled by a computer or computers (120). Thus, in certain embodiments, the present disclosure may comprise software for controlling the various components of the system, including pumps, valves, autosamplers, and the like. Such software can be used to optimize the extraction process through the precise timing of sample and solute additions and flow rate.

Although some or all of the steps in the method and the stations comprising the system may be on-line, in certain embodiments, some or all of the steps may be performed “off-line.” In contrast to the term “on-line”, the term “off-line” refers to a purification, separation, or extraction procedure that is performed separately from previous and/or subsequent purification or separation steps and/or analysis steps. In such off-line procedures, the analytes of interests typically are separated, for example, on an extraction column or by liquid/liquid extraction, from the other components in the sample matrix and then collected for subsequent introduction into another chromatographic or detector system. Off-line procedures typically require manual intervention on the part of the operator.

Kits

In certain embodiments, the disclosure provides kits for use in accordance with methods and compositions disclosed herein. Generally, kits comprise one or more reagents detect the biomarker or biomarkers of interest. In an embodiment, at least one of the reagents (e.g., primers, probes antibody or other binding agent) is labeled with a detectable moiety. Suitable reagents may include nucleic acid probes and/or antibodies or fragments thereof. In some embodiments, suitable reagents are provided in a form of an array such as a microarray or a mutation panel.

In some embodiments, provided kits further comprise reagents for carrying out at least one of the various detection methods described herein (e.g., quantitative PCR and/or real-time PCR, sequencing, hybridization, primer extension, multiplex ASPE, immunoassays, etc.). For example, kits may optionally contain buffers, enzymes, and/or reagents for use in methods described herein, e.g., for quantitative PCR and/or real-time PCR, amplifying nucleic acids via primer-directed amplification, for performing ELISA experiments.

In some embodiments, provided kits further comprise a control indicative of a healthy individual, e.g., a nucleic acid and/or protein sample from an individual who does not have kidney fibrosis. Additionally and/or alternatively, in some embodiments, provided kits further comprise a positive disease control indicative of an individual afflicted with kidney fibrosis. Or, positive controls containing known amounts of the biomarker of interest and/or negative controls having no biomarker may be included. Kits may also contain instructions on how to determine if an individual has the disease and/or syndrome of interest, or is at risk of developing the disease and/or syndrome of interest.

In some embodiments, provided is a computer readable medium encoding information corresponding to the biomarker of interest. Such computer readable medium may be included in a kit of the invention.

Methods to Identify Kidney Fibrosis biomarkers

Data Mining

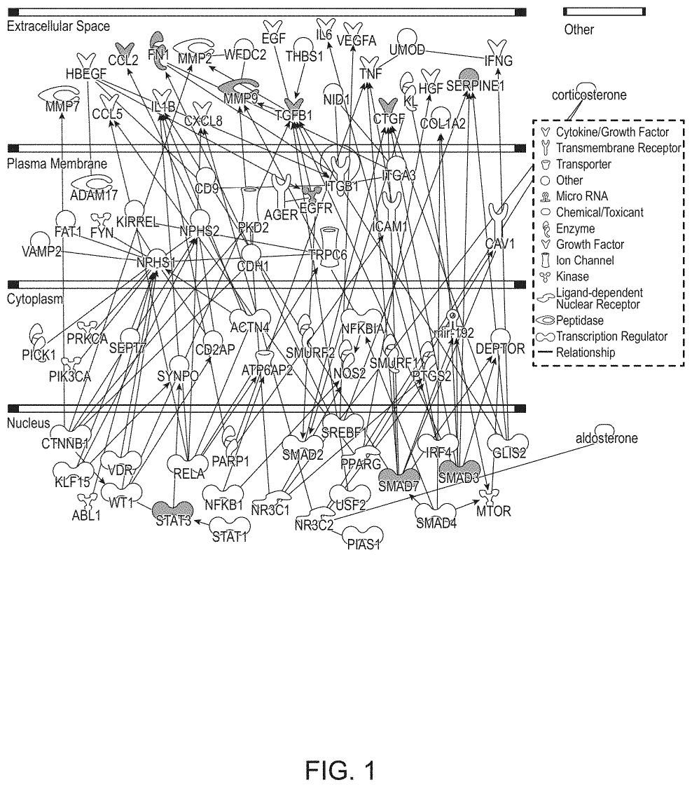

In certain embodiments of the disclosure, biomarkers are identified using a data mining approach. See e.g., FIGS. 1, 2 and 3 show biomarkers identified as being associated with kidney fibrosis using such an approach. For example, in some cases public databases (e.g., PubMed) may be searched for genes that have been shown to be linked to (directly or indirectly) to a certain disease. This approach is described in U.S. patent application Ser. No. 15/977,000, filed May 11, 2018, the disclosure of which is incorporated by reference in its entirety herein. Such genes may then be evaluated as biomarkers.

Disease Indication Model Construction

Disease models (e.g., gene/protein-gene/protein interaction networks) based on published peer-reviewed research were constructed to simulate disease biology using an integrated software suite for functional analysis of Next Generation Sequencing, variant, CNV, microarray, metabolic, SAGE, proteomics, siRNA, microRNA, and screening data. The resulting output was a single interaction network for the gene set associated with kidney fibrosis. The indication model was generated tissue specific: the model was constructed using genes expressed in the kidney (e.g., kidney parenchyma).

The model included genes/proteins and interactions between them. An example of such a model is shown in FIG. 1. These genes/proteins included both the initial objects or “seeds” (i.e., the 57 highly relevant genes/proteins identified from the data mining, which was ultimately restricted to 26 genes/proteins using the tissue based modeling approach) and 54 secondary genes/proteins that were identified in the modeling that link the initial objects. Additionally, two potential candidate test/biomarkers not genes/proteins but chemicals or small molecules (corticosterone and aldosterone [corticosterone is the precursor molecule to the mineralocorticoid aldosterone, one of the major homeostatic modulators of sodium and potassium levels in vivo]) known to be involved with kidney fibrosis by way of published peer-reviewed research were included in the model construction. Indication modeling simulates the protein-protein-chemical (small molecule) interaction “neighborhood” in a specific tissue around genes directly associated with the indication under investigation. The nature of the biomarker (i.e., cytokine, miRNA, transcription regulator) is shown in the box to the right.

Model Validation

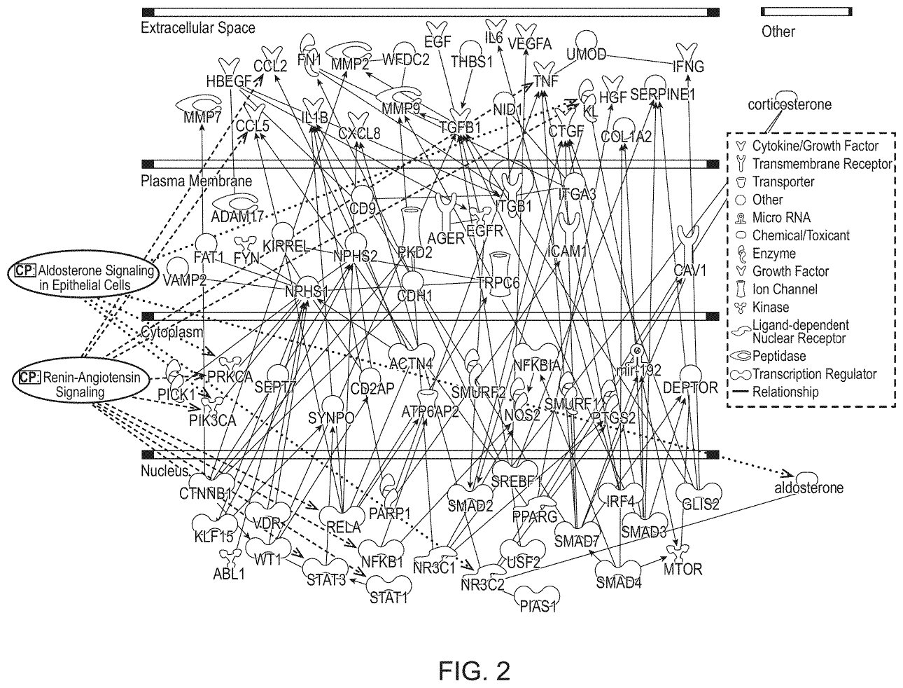

The indication modeling is an iterative process that includes validation to ensure the modeling accurately simulates disease biology. A statistical approach was taken to validate the model and verify enrichment of genes from the model in an independent third party data source. As shown in FIG. 2, the indication modeling accurately captured much of the known biological pathways including components of the renin-angiotensin-aldosterone system and aldosterone signaling in epithelial cells, which is indicative of the potential beneficial effects from including the chemicals or small molecules corticosterone and aldosterone in the indication modeling.

Ranking Candidate Biomarkers

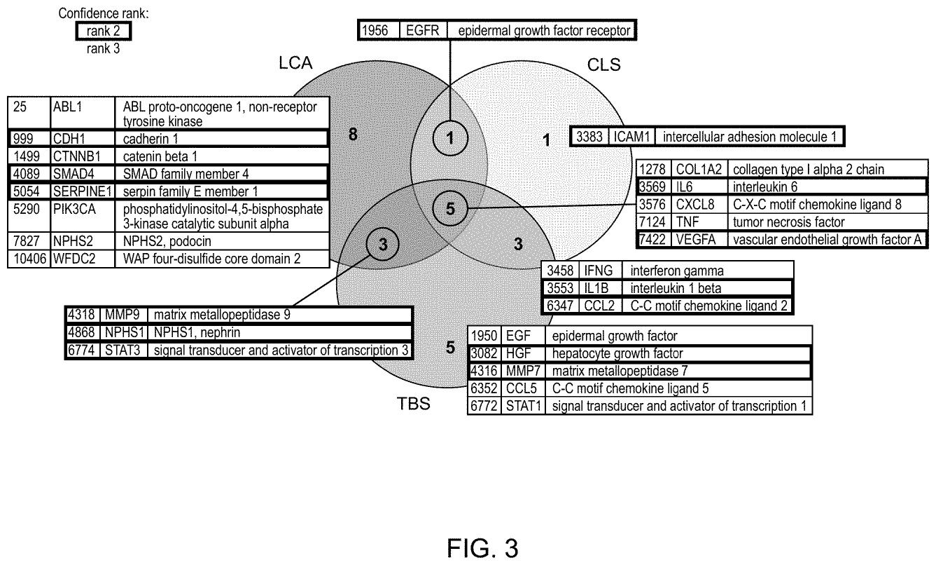

A stepwise process was used to provide a confidence score for candidate biomarkers. Rank 1 candidate biomarkers (i.e., highest confidence candidates) were those genes, proteins, or chemicals independently recommended by one or more therapeutic experts as a “biomarker” for the indication network modeled. Rank 2 candidate biomarkers (i.e. lower confidence candidates) were those genes, proteins, or chemicals identified by data mining for the indication network modeled, for example, genes or proteins specific to the indicated disease of renal fibrosis identified via MEDLINE data mining. Rank 3 candidate biomarkers (i.e. lowest confidence candidates) were the genes or proteins that are not Rank 1 or Rank 2, for example, additional genes or proteins obtained via modeling based on triplets (FIG. 1). FIG. 3 shows an example of such an analysis for Rank 2 and 3 biomarkers. Rank 1 biomarkers are shown as shaded symbols in FIG. 1.

In one embodiment, the results of such an analysis identify at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2), nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA). Or, the method may identify at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or all 31 of the biomarkers as being highly associated with kidney fibrosis. In certain embodiments, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2). In certain embodiments, the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7). In certain embodiments, the biomarker comprises at least one of transforming growth factor beta 1 (TGFB1) or metallopeptidase 2 (MMP2). Additionally and/or alternatively, the method may include identification of at least one normalization (e.g., housekeeping gene).

Molecular

In certain embodiments, the disclosure comprises methods to identify biomarkers for a syndrome or disease of interest (i.e., variants in nucleic acid sequence that are associated with kidney fibrosis in a statistically significant manner). The genes and/or genomic regions assayed for new biomarkers may be selected based upon their importance in biochemical pathways that show genetic linkage and/or biological causation to the syndrome and/or disease of interest. Or, the genes and/or genomic regions assayed for biomarkers may be selected based on genetic linkage to DNA regions that are genetically linked to the inheritance of kidney fibrosis in families. Or, the genes and/or genomic regions assayed for biomarkers may be evaluated systematically to cover certain regions of chromosomes not yet evaluated.

In other embodiments, the genes or genomic regions evaluated for new biomarkers may be part of a biochemical pathway that may be linked to the development of kidney fibrosis. The variants and/or variant combinations may be assessed for their clinical significance based on one or more of the following methods. If a variant or a variant combination is reported or known to occur more often in nucleic acid from subjects with, than in subjects without kidney fibrosis or associated pathologies it is considered to be at least potentially predisposing to kidney fibrosis. If a variant or a variant combination is reported or known to be transmitted exclusively or preferentially to individuals having kidney fibrosis, it is considered to be at least potentially predisposing to kidney fibrosis. Conversely, if a variant is found in both populations at a similar frequency, it is less likely to be associated with the development of kidney fibrosis.

If a variant or a variant combination is reported or known to have an overall deleterious effect on the function of a protein or a biological system in an experimental model system appropriate for measuring the function of this protein or this biological system, and if this variant or variant combination affects a gene or genes known to be associated with the syndrome and/or disease of interest, it is considered to be at least potentially predisposing to the syndrome and/or disease of interest. For example, if a variant or a variant combination is predicted to have an overall deleterious effect on a protein or gene expression (i.e., resulting in a nonsense mutation, a frameshift mutation, or a splice site mutation, or even a missense mutation), based on the predicted effect on the sequence and/or the structure of a protein or a nucleic acid, and if this variant or variant combination affects a gene or genes known to be associated with kidney fibrosis and/or associated pathologies, it is considered to be at least potentially predisposing to the syndrome and/or disease of interest.

Also, in certain embodiments, the overall number of variants may be important. If, in the test sample, a variant or several variants are detected that are, individually or in combination, assessed as at least probably associated with kidney fibrosis, then the individual in whose genetic material this variant or these variants were detected can be diagnosed as being affected with or at high risk of developing kidney fibrosis.

For example, the disclosure herein provides methods for diagnosing the presence or an increased risk of developing kidney fibrosis in a subject. Such methods may include obtaining a nucleic acid from a sample of tissue or body fluid. The method may further include sequencing the nucleic acid or determining the genomic arrangement or copy number of the nucleic acid to detect whether there is a variant or variants in the nucleic acid sequence or genomic arrangement or copy number. The method may further include the steps of assessing the clinical significance of a variant or variants. Such analysis may include an evaluation of the extent of association of the variant sequence in affected populations (i.e., subjects having the disease). Such analysis may also include an analysis of the extent of the effect the mutation may have on gene expression and/or protein function. The method may also include diagnosis the presence or an increased risk of developing kidney fibrosis based on the assessment. The following examples serve to illustrate certain aspects of the disclosure. These examples are in no way intended to be limiting.

### EXAMPLES

### Example 1

### Biomarkers for Kidney Fibrosis

Biomarkers disclosed herein may be measured using any of the techniques discussed above or in some cases, by one of the non-limiting assays disclosed below.

Transforming Growth Factor Beta 1 (TGFβ1)

Transforming growth factor beta 1 is a polypeptide member of the transforming growth factor beta superfamily of cytokines. It is encoded by the TGF β1 gene. It is a secreted protein that performs many cellular functions, including the control of cell growth, proliferation, differentiation and apoptosis. TGF β1 can be measured by immunoassay (see e.g., Kropf et al., Clinical Chemistry, 1997, 43:1965-74).

Uromodulin (UMOD)

Uromodulin also known as Tamm-Horsfall glycoprotein (THP), is a glycoprotein that in humans is encoded by the UMOD gene Uromodulin is the most abundant protein excreted in urine. Uromodulin can be measured by ELISA (e.g., ThermoFisher Scientific) or other methods known in the art.

Connective Tissue Growth Factor (CTGF)

Connective tissue growth factor, is a matricellular protein of the CCN family of extracellualr matrix-associated heparin binding proteins. CTGF has important roles cell adhesion and migration, cell proliferation and angiogenesis, and is involved in fibrotic disease. CTGF may be measured by ELISA (e.g., BioVendor Research and Diagnostic Products) or other methods known in the art.

EGFR

Epidermal growth factor receptor (EGFR) is a transmembrane receptor protein in humans. EGFR expression levels are measured by fluorescent in situ hybridization (FISH) using 4-5 micron FFPE sections of tissue. Alternatively, expression may be measured in serum, plasma, urine, bone marrow, blood, cerebrospinal fluid, FNA, or bone marrow. Gene mutation analysis is performed using real-time PCR, single base extension and/or DNA sequencing. Gene mutation can be determined in, for examples, tissue, serum, plasma or urine.

Cadherin (CDH1), SMAD family members (e.g., SMAD4), Cadherin-1 also known as CAM 120/80 or epithelial cadherin (E-cadherin) is a protein encoded by the CDH1 gene. CDH1 has also been designated as CD324 (cluster of differentiation 324) and is a tumor suppressor.

SMADs comprise a family of structurally similar proteins that are the main signal transducers for receptors of the transforming growth factor beta (TGF-B) superfamily, which are critically important for regulating cell development and growth. There are three distinct sub-types of SMADs: receptor-regulated SMADs (RSMADs), common partner SMADs (Co-SMADs, and inhibitory SMADs (I-SMADs). The eight members of the Smad family are divided among these three groups. Trimers of two receptor-regulated SMADs and one co-SMAD act as transcription factors that regulate the expression of certain genes

These genes related to hereditary cancer is examined by next generation sequencing analysis. Additionally, portions of the flanking noncoding regions are also examined. Comprehensive deletion/ duplication testing is performed using microarray CGH for 20 genes, and by multiplex ligation-dependent probe amplification (MLPA) for the CHEK2 and PMS2 genes. Genes tested in this panel may include APC, ATM, AXIN2, BLM, BMPR1A, BRCA1, BRCA2, CDH], CDKN2A, CHEK2, EPCAM, MLH1, MSH2, MSH6, MUTYH, PMS2, POLD1, POLE, PTEN, SMAD4, STK11 and TP53. Or, additional genes (e.g., SMAD2, SMAD3, SMAD7) may be added. Clinically significant findings are confirmed by Sanger sequencing or qPCR. Results are reported using ACMG guidelines and nomenclature recommended by the Human Genome Variation Society (HGVS).

Galectin-1 and Galectin-3

Galectin- and Galectin-3 are measured by ELISA or other commercially available immunoassay systems. See e.g., Alam et al., Kidney Int. Rep., 2018, 21:103-111; Al-Obaidi et al., FASEB J., 2019, 33:373-387; Tan et al., Diabetologia, 2018, 61:1212-1219; de Boer et al., BMJ Open Diabetes Res. Care, 2017, Nov. 14; 5(1):e000461; Rebholz et al., Kidney Int., 2018, 93:252-259; Martinez-Martinez et al., J. Hypertens., 2018, 36:368-376.

Serpin family E member 1 (SERPINE1)

Plasminogen activator inhibitor-1 (PAI-1) also known as endothelial plasminogen activator inhibitor or serpin E1 is a protein that in humans is encoded by the SERPINE1 gene. Elevated PAI-1 is a risk factor for thrombosis and atherosclerosis. PAI-1 is a serine protease inhibitor (serpin) that functions as the principal inhibitor of tissue plasminogen activator (tPA) and urokinase (uPA), activators of plasminogen and fibrinolysis. It is a serine protease inhibitor (serpin) protein (SERPINE1). The PAI-1 gene is SERPINE1, located on chromosome 7 (7q21.3-q22). There is a common polymorphism known as 4G/5G in the promoter region. The 5G allele is slightly less transcriptionally active than the 4G.

Metallopeptidases (MMP2, MMP7, and MMP9)

Proteins of the matrix metallowproteinnase (MMP) family are involved in the breakdown of extracellular matrix (ECM) in normal physiological processes, such as embryonic development, reproduction, and tissue remodeling, as well as in disease processes, such as arthritis and metastasis. Most MMP's are secreted as inactive preproteins which are activated when cleaved by extracellular proteinases.

Activation of MMP-2 (encoded by MMP2 gene) requires proteolytic processing. A complex of membrane type 1 MMP (MT1-MMP/MMP14) and tissue inhibitor of metalloproteinase 2 recruits pro-MMP 2 from the extracellular milieu to the cell surface. Activation then requires an active molecule of MT1-MMP and auto catalytic cleavage. Clustering of integrin chains promotes activation of MMP-2.

Matrix metalloproteinase-7 (encoded by MMP7 gene) is an enzyme in humans that is encoded by the MMP7 gene Proteins of the matrix metalloproteinase (MMP) family are involved in the breakdown of extracellular matrix in normal physiological processes, such as embryonic development, reproduction, and tissue remodeling, as well as in disease processes, such as arthritis and metastasis. MMP-7 degrades proteoglycans, fibronectin, elastin and casein. MMP7 is involved in wound healing and has been implicated in the regulation of defensins in intestinal mucosa.

MMP-9 (encoded by MMP9 gene) is a biomarker of inflammation, tissue remodeling, wound healing, and mobilization of tissue-bound growth factors and cytokines. Its expression correlates with abnormal collagen deposition. MMP-9 contributes to the pathogenesis of numerous clinical disease states, including rheumatic arthritis, coronary artery disease, chronic obstructive pulmonary disease, multiple sclerosis, asthma, and cancer. MMPs are synthesized as inactive zymogens, and must be enzymatically activated by hydrolytic cleavate of a pro-peptide domain. MMPs are measured by enzyme-linked immunoabsorbant assay (ELISA), gel zymography, substrate assays, or in situ zymography. In gel zymography, proteins are separated by electrophoresis, using a non-reducing SDS-PAGE gel that is embedded with gelatin. After separation, SDS is removed from the gel, and the gel is submerged in a solution that contains essential cofactors required for enzyme activity. MMPs within the gel digest the gelatin. resulting in clear bands on a dark blue background after staining with Coomassie Blue. For in situ zymog raphy, tissue sections are bathed with a fluorescent substrate peptide. As the fluorescent substrate is hydrolyzed by MMP-9 present in the tissue section, loss of fluorescence is seen as black holes on a fluorescent background (ABCAM).

Nephrin (NPHS1) and Podocin (NPHS2)

Nephrin, encoded by the NPHS1 gene, is a protein necessary for the proper functioning of the renal filtration barrier. Podocin is encoded by the NPHS2 gene. The renal filtration barrier consists of fenestrated endothelial cells, the glomerular basement membrane, and the podocytes of epithelial cells. Nephrin is a transmembrane protein that is a structural component of the slit diaphragm, on the tips of the podocytes. NPHS1 has an extracellular domain that contains eight distal IgG like domains and one proximal fibronectin type III domain, a transmembrane domain and a short intracellular domain. NPHS1 molecules show both homophilic and heterophilic interactions. Among heterophilic interaction partners, slit diaphragm proteins such as Kin of IRRE-like protein 1 (KIRREL, Nephrin-like protein 1, NEPH1), KIRREL3 (NEPH2) and KIRREL2 (NEPH3) were shown to stabilize the slit diaphragm structure. Intracellularly Podocin (NPHS2), CD2 associated protein (CD2AP) and adherins junction associated proteins like IQGAP, MAGI, CASK and spectrins all interact with NPHS1. Nephrin may play a major role in organizing the molecular structure of the slit diaphragm itself and via its binding partners links it to the actin cytoskeleton. Mutations of NPHS1 or NPHS2, which can be detected by DNA sequencing, can lead to early onset of heavy proteinuria and rapid progression to end-stage renal disease.

Signal transducer and activator of transcription 3 (STAT3)

Signal transducer and activator of transcription 3 (encoded by STA 13 gene) is a member of the STAT protein family. In response to cytokines and growth factors, STAT3 is phosphorylated by receptor-associated Janus kinases (JAK), form homo- or heterodimers, and translocate to the cell nucleus where they act as transcription activators. Mutatations in STAT3 are detected by DNA sequencing of all coding nucleotides of gene STAT3, plus at least two and typically 20 flanking intronic nucleotides upstream and downstream of each coding exon, covering the conserved donor and acceptor splice sites, as well as typically 20 flanking nucleotides in the 5′ and 3′ UTR.

Intercellular adhesion molecule 1 (ICAM1)

ICAM-1 (Intercellular Adhesion Molecule 1) also known as CD54 (Cluster of Differentiation 54) and encoded by the /CAM/ gene, is a protein that in humans is encoded by the ICAM1 gene. ICAM-1 is a member of the immunoglobulin superfamily. This cell surface glycoprotein is typically expressed on endothelial cells and cells of the immune system, and binds to integrins of type CD11a/CD18, or CD11b/CD18. ICAM-1 can be measure by immunoassay (e.g., Pacific Biomarkers).

Interleukin 6 (IL6)

Interleukin 6 (IL-6) encoded by the IL6 gene, is an interleukin that acts as both a pro-inflammatory cytokine and an anti-inflammatory myokine. In humans, it is encoded by the IL6 gene. Interleukin 6 is secreted by T cells and macrophages to stimulate immune response, e.g. during infection and after tissue damage leading to inflammation. IL-6 also plays a role in fighting infection. In addition, osteoblasts secrete IL-6 to stimulate osteoclast formation. Smooth muscle cells in the tunica media of many blood vessels also produce IL-6 as a pro-inflammatory cytokine. IL-6's role as an anti-inflammatory cytokine is mediated through its inhibitory effects on TNF-alpha and IL-1, and activation of IL-1ra and IL-10. IL-6 is an important mediator of fever and of the acute phase response. It is capable of crossing the blood-brain barrier, initiating synthesis of PGE2 in the hypothalamus, and changing the body's temperature setpoint. In muscle and fatty tissue, IL-6 stimulates energy mobilization that leads to increased body temperature. IL-6 can be secreted by macrophages in response to specific microbial molecules, referred to as pathogen-associated molecular patterns (PAMPs), and is also considered a myokine (a cytokine produced from muscle), which is elevated in response to muscle contraction. IL6 can be measured by a biological assay (see e.g., Nordan et al., Curr. Protoc. Immunol., 2001) or by ELISA.

Vascular Endothelial Growth Factor A (VEGFA)

Vascular endothelial growth factor (VEGF), also known as vascular permeability factor (VPF), is a homodimeric 34 to 45 kilodalton, heparin-binding glycoprotein. VEGF has potent angiogenic, mitogenic, and vascular permeability-enhancing activities specific for endothelial cells. VEGF is thought to play an important role in several physiologic processes, including wound healing, ovulation, menstruation, maintenance of blood pressure, and pregnancy. VEGF has also been associated with a number of pathologic processes that involve angiogenesis, including arthritis, psoriasis, macular degeneration, and diabetic retinopathy. Also, tumor expression of proangiogenic factors, including VEGF, has been associated with advanced tumor progression in a number of human cancers. VEGF can be measured in serum using enzyme immunoassay (EIA).

Interleukin 1 beta (IL1B)

Interleukin 1 beta (IL1β) encoded by the IL1B gene, is a cytokine protein that in humans is encoded by the IL1B gene. There are two genes for interleukin-1 (IL-1): IL-1 alpha and IL-1 beta. IL-1β precursor is cleaved by cytosolic caspase 1 (interleukin 1 beta convertase) to form mature IL-1β. IL-1β is a member of the interleukin 1 family of cytokines. This cytokine is produced by activated macrophages as a proprotein, which is proteolytically processed to its active form by caspase 1 (CASP1/ICE). This cytokine is an important mediator of the inflammatory response, and is involved in a variety of cellular activities, including cell proliferation, differentiation, and apoptosis. Increased production of IL-10 causes a number of different autoinflammatory syndromes. IL-1 beta can be measured by RIA or other immunoassays (see e.g., Endres et al., Clin. Immunol Immunopathol., 1988, 49:424-38).

C—C Motif Chemokine Ligand 2 (CCL2)

The chemokine (C—C motif) ligand 2 (encoded by the CCL2 gene) is also referred to as monocyte chemoattractant protein 1 (MCP1) and small inducible cytokine A2. CCL2 is a small cytokine that belongs to the CC chemokine family. CCL2 recruits monocytes, memory T cells, and dendritic cells to the sites of inflammation produced by either tissue injury or infection. Administration of anti-CCL2 antibodies in a model of glomerulonephritis reduces infiltration of macrophages and T cells, reduces crescent formation, as well as scarring and renal impairment. Hypomethylation of CpG sites within the CCL2 promoter region is affected by high levels of blood glucose and TG, which increase CCL2 levels in the blood serum. The later plays an important role in the vascular complications of type 2 diabetes. CCL2 can be measured by ELISA (Cisbio).

Hepatocyte Growth Factor (HGF)

Hepatocyte growth factor, encoded by the HGF gene, regulates cell growth, cell motility, and morphogenesis. Hepatocyte growth factor is secreted by mesenchymal cells and acts as a multi-functional cytokine on cells of mainly epithelial origin. Its ability to stimulate mitogenesis, cell motility, and matrix invasion gives it a central role in angiogenesis, tumorogenesis, and tissue regeneration. Hepatocyte growth factor can be measured by ELISA (Yamanougchi et al., Resp. Med., 1998, 92:273-278).

Corticosterone and Aldosterone

Coriticosterone is measured by LC-MS/MS from frozen samples of serum. Aldosterone is measured by LC-MS/MS from serum or plasma; samples do not need to be frozen.

ADAM metalloproteinase domain 17 (ADAM17)

ADAM metallopeptidase domain 17 (ADAM17), also called TACE (tumor necrosis factor-a-converting enzyme), is a 70-kDa enzyme that belongs to the ADAM protein family of disintegrins and metalloproteinases. Quantitative PCR may be used to measure mRNA levels of ADAM17. An ELISA may be used to measure ADAM17 protein levels.

Fibronectin 1 (FN1)

Fibronectin is a high-molecular weight (˜440 kDa) glycoprotein of the extracellular matrix that binds to membrane-spanning receptor proteins called integrins. Fibronectin also binds to other extracellular matrix proteins such as collagen, fibrin, and heparan sulfate proteoglycans (e.g. syndecans)

Kruppel like factor 15 (KLF15)

Kruppel-like factor 15 is a proten encoded by the KLF15 gene in the Kruppel-like factor family. Its former designation KKLF stands for kidney-enriched Kruppel-like factor. Expression levels may be measure by Real-time PCR (see e.g., Mallipattu et al., J. Biol. Chem, 2012, 287:19122-35)

Nuclear factor kappa B subunit 1 (NFKB1)

Nuclear factor NF-kappa-B p105 subunit is a protein that in humans is encoded by the NFKB1 gene. The 105 kD protein can undergo cotranslational processing by the 26S proteasome to produce a 50 kD protein. The 105 kD protein is a Rel protein-specific transcription inhibitor and the 50 kD protein is a DNA binding subunit of the NF-kappaB protein complex. Activated NF-κB translocates into the nucleus and stimulates the expression of genes involved in a wide variety of biological functions; over 200 known genes are targets of NF-κB in various cell types, under specific conditions. Inappropriate activation of NF-κB has been associated with a number of inflammatory diseases while persistent inhibition of NF-κB leads to inappropriate immune cell development or delayed cell growth. Nuclear factor kappa B subunit 1 can be measured by real time PCR.

Nuclear Receptor Subfamily 3 Group C Member 2 (NR3C2)

This gene encodes the mineralocorticoid receptor, which mediates aldosterone actions on salt and water balance within restricted target cells. The protein functions as a ligand-dependent transcription factor that binds to mineralocorticoid response elements in order to transactivate target genes. Mutations in this gene cause autosomal dominant pseudohypoaldosteronism type I, a disorder characterized by urinary salt wasting. Defects in this gene are also associated with early onset hypertension with severe exacerbation in pregnancy.

### Example 2

### Embodiments

The disclosure may be better understood by reference to the following non-limiting embodiments.
A1. A method to detect a biomarker associated with kidney fibrosis in an individual comprising the steps of:

obtaining a biological sample from the individual; and

measuring the amount of the biomarker, and/or the amount of or a mutation in a nucleic acid (e.g. genomic DNA or mRNA) that encodes for, or regulates expression of the biomarker in the biological sample.
A.2. The method of any one of the previous and/or subsequent embodiments, wherein the biomarker comprises at least one of transforming growth factor beta 1 (TGFB1) or metallopeptidase 2 (MMP2).
A.3. The method of any one of the previous and/or subsequent embodiments, wherein the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7).
A.4. The method of any one of the previous and/or subsequent embodiments, wherein, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2).
A.5. The method of any one of the previous and/or subsequent embodiments, wherein the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2) nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA).
A.6. The method of any one of the previous and/or subsequent embodiments, wherein the measuring comprises measurement of protein.
A.7. The method of any one of the previous and/or subsequent embodiments, wherein the measuring comprises analysis of nucleic acid sequence or expression.
A.8. The method of any one of the previous and/or subsequent embodiments, wherein the biological sample comprises a liquid or tissue biopsy, cell-free nucleic acid, blood, urine, serum or plasma.
A.9. The method of any one of the previous and/or subsequent embodiments, wherein the measuring comprises an immunoassay.
A.10. The method of any one of the previous and/or subsequent embodiments, wherein the measuring comprises flow cytometry.
A.11. The method of any one of the previous or subsequent embodiments, wherein the measuring comprises mass spectrometry or liquid chromatography tandem mass spectrometry (LC-MS/MS).
A.12. The method of any one of the previous and/or subsequent embodiments, wherein at least 5 of the biomarkers are measured.
B.1. A method of identifying a biomarker associated with kidney fibrosis in an individual comprising: identifying a biomarker having increased or decreased expression in an individual having or suffering from kidney fibrosis as compared to normal controls.
B.2. A method to detect the presence of, or susceptibility to, kidney fibrosis in an individual comprising:

obtaining a biological sample from the individual;

measuring the amount of the biomarker, and/or the amount of or a mutation in a nucleic acid (e.g. genomic DNA or mRNA) that encodes for, or regulates expression of a biomarker; and

comparing the amount of, and/or the amount or a mutation in a nucleic acid that encodes for, or regulates expression of the biomarker in the biological sample with a control value for the biomarker.
B.3. The method of any one of the previous and/or subsequent embodiments, wherein the biomarker comprises at least one of transforming growth factor beta 1 (TGFB1) or metallopeptidase 2 (MMP2).
B.4. The method of any one of the previous and/or subsequent embodiments, wherein the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7).
B.5. The method of any one of the previous and/or subsequent embodiments, wherein, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2).
B.6. The method of any one of the previous and/or subsequent embodiments, wherein the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2) nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA).
B.7. The method of any of the previous and/or subsequent embodiments, wherein the measuring comprises measurement of protein.
B.8. The method of any of the previous and/or subsequent embodiments, wherein the measuring comprises analysis of nucleic acid sequence or expression.
B.9. The method of any of the previous and/or subsequent embodiments, wherein the biological sample comprises a liquid or tissue biopsy, cell-free nucleic acid, blood, urine, serum or plasma.
B.10. The method of any of the previous and/or subsequent embodiments, wherein the measuring comprises an immunoassay.
B.11. The method of any of the previous and/or subsequent embodiments, wherein the measuring comprises flow cytometry.
B.12. The method of any of the previous and/or subsequent embodiments, wherein the measuring comprises mass spectrometry or liquid chromatography tandem mass spectrometry
B.13. The method of any one of the previous and/or subsequent embodiments, wherein at least 5 of the biomarkers are measured.
C.1. A composition to detect a biomarker associated with kidney fibrosis in an individual comprising reagents that measure the amount of the biomarker, and/or the amount or a mutation in a nucleic acid (e.g. genomic DNA or mRNA) that encodes for, or regulates expression of the biomarker.
C.2. The composition of any one of the previous and/or subsequent embodiments, wherein the biomarker comprises at least one of transforming growth factor beta 1 (TGFB1) or metallopeptidase 2 (MMP2).
C.3. The composition of any one of the previous and/or subsequent embodiments, wherein the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7).
C.4. The composition of any one of the previous and/or subsequent embodiments, wherein the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2).
C.5. The composition of any one of the previous and/or subsequent embodiments, wherein the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2) nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA).
C.6. The composition of any of the previous and/or subsequent embodiments, wherein the measuring comprises measurement of protein.
C.7. The composition of any of the previous and/or subsequent embodiments, wherein the measuring comprises analysis of nucleic acid sequence or expression.
C.8. The composition of any of the previous and/or subsequent embodiments, wherein the biological sample comprises a liquid or tissue biopsy, cell-free nucleic acid, blood, urine, serum or plasma.
C.9. The composition of any of the previous and/or subsequent embodiments, wherein the measuring comprises an immunoassay.
C.10. The composition of any of the previous and/or subsequent embodiments, wherein the measuring comprises flow cytometry.
C.11. The composition of any of the previous and/or subsequent embodiments, wherein the measuring comprises mass spectrometry or liquid chromatography tandem mass spectrometry (LC-MS/MS).
C.12. A composition comprising any of the previous and/or subsequent embodiments for measuring at least 5 of the biomarkers.
C.13. A composition comprising any of the previous and/or subsequent embodiments wherein at least one reagent is labeled with a detectable moiety.
D.1. A kit comprising reagents to detect a biomarker associated with kidney fibrosis in an individual comprising: reagents that measure the amount of the biomarker, and/or the amount of, or a mutation in, a nucleic acid (e.g. genomic DNA or mRNA) that encodes for, or regulates expression of the biomarker; and instructions for use.
D.2. The kit of any one of the previous and/or subsequent embodiments, wherein the biomarker comprises at least one of transforming growth factor beta 1 (TGFB1) or metallopeptidase 2 (MMP2).
D.3. The kit of any one of the previous and/or subsequent embodiments, wherein the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7).
D.4. The kit of any one of the previous and/or subsequent embodiments, wherein, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2).
D.5. The kit of any one of the previous and/or subsequent embodiments, wherein the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2) nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA).
D.6. The kit of any of the previous and/or subsequent embodiments, wherein the measuring comprises measurement of protein.
D.7. The kit of any of the previous and/or subsequent embodiments, wherein the measuring comprises analysis of nucleic acid sequence or expression.
D.8. The kit of any of the previous and/or subsequent embodiments, wherein the biological sample comprises a liquid or tissue biopsy, cell-free nucleic acid, blood, urine, serum or plasma.
D.9. The kit of any of the previous and/or subsequent embodiments, wherein the measuring comprises an immunoassay.
D.10. The kit of any of the previous and/or subsequent embodiments, wherein the measuring comprises flow cytometry.
D.11. The kit of any of the previous and/or subsequent embodiments, wherein the measuring comprises mass spectrometry or liquid chromatography tandem mass spectrometry (LC-MS/MS).
D.12. A kit comprising any of the previous and/or subsequent embodiments for measuring at least 5 of the biomarkers.
D.13. A kit comprising any of the previous and/or subsequent embodiments wherein at least one reagent is labeled with a detectable moiety.
E.1. A system to detect a biomarker associated with kidney fibrosis in an individual.
E.2. The system of any of the previous and/or subsequent embodiments, comprising a station for providing a sample believed to contain the biomarker; optionally, a station for separating the biomarker from other components in the sample; a station for measuring the amount of the biomarker; and a station to analyze the results to determine the presence or amount of the biomarker in the sample.
E.3. The system of any of the previous and/or subsequent embodiments, wherein at least one of the stations is automated and/or controlled by a computer.
E.4. The system of any of the previous and/or subsequent embodiments, wherein the measurement comprises measurement of the amount of the biomarker, and/or the amount of, or a mutation in, a nucleic acid (e.g. genomic DNA or mRNA) that encodes for, or regulates expression of the biomarker.
E.5. The system of any one of the previous and/or subsequent embodiments, wherein the biomarker comprises at least one of transforming growth factor beta 1 (TGFB1) or metallopeptidase 2 (MMP2).
E.6. The system of any one of the previous and/or subsequent embodiments, wherein the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7).
E.7. The system of any one of the previous and/or subsequent embodiments, wherein, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2).
E.8. The system of any one of the previous and/or subsequent embodiments, wherein the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2) nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA).
E.9. The methods, compositions, kit or system of any of the previous and/or subsequent embodiments, wherein the housekeeping gene is glyceraldehyde 3-phosphate dehydrogenase.
E.10. The system of any of the previous and/or subsequent embodiments, wherein the measuring comprises measurement of protein.
E.11. The system of any of the previous and/or subsequent embodiments, wherein the measuring comprises analysis of nucleic acid sequence or expression.
E.12. The system of any of the previous and/or subsequent embodiments, wherein the biological sample comprises a liquid or tissue biopsy, cell-free nucleic acid, blood, urine, serum or plasma.
E.13. The system of any of the previous and/or subsequent embodiments, wherein the measuring comprises an immunoassay.
E.14. The system of any of the previous and/or subsequent embodiments, wherein the measuring comprises flow cytometry.
E.15. The system of any of the previous and/or subsequent embodiments, wherein the measuring comprises mass spectrometry or liquid chromatography tandem mass spectrometry (LC-MS/MS)
E.16. A system comprising any of the previous and/or subsequent embodiments for measuring at least 5 of the biomarkers.
F.1. A method of treating an individual having or susceptible to developing kidney fibrosis comprising identifying a biomarker associated with kidney fibrosis in an individual having or suffering from kidney fibrosis comprising: identifying a biomarker having increased or decreased expression in kidney fibrosis as compared to normal controls.
F.2. A method of treating an individual having or susceptible to developing kidney fibrosis kidney fibrosis comprising:

obtaining a sample from the individual;

measuring the amount of the biomarker, and/or the amount of or a mutation in a nucleic acid (e.g. genomic DNA or mRNA) that encodes for, or regulates expression of a biomarker in the sample;

comparing the amount of the biomarker, and/or the amount or a mutation in a nucleic acid that encodes for, or regulates expression of at least one of the expression of the biomarker in the sample with a control value the biomarker; and

treating the individual for kidney fibrosis or to reduce the rate of developing kidney fibrosis when a difference between gene expression in the individual and the control value indicates that the individual may have (i.e., is diagnostic of the presence of), or is susceptible to developing (i.e., is at increased risk for) kidney fibrosis.
F.3. The method of any one of the previous and/or subsequent embodiments, wherein the biomarker comprises at least one of transforming growth factor beta 1 (TGFB1) or metallopeptidase 2 (MMP2).
F.4. The method of any one of the previous and/or subsequent embodiments, wherein the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7).
F.5. The method of any one of the previous and/or subsequent embodiments, wherein, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2).
F.6. The method of any one of the previous and/or subsequent embodiments, wherein the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2) nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA).
F.7. The method of any of the previous and/or subsequent embodiments, wherein the measuring comprises measurement of protein.
F.8. The method of any of the previous and/or subsequent embodiments, wherein the measuring comprises analysis of nucleic acid sequence or expression.
F.9. The method of any of the previous and/or subsequent embodiments, wherein the biological sample comprises a liquid or tissue biopsy, cell-free nucleic acid, blood, urine, serum or plasma.
F.10. The method of any of the previous and/or subsequent embodiments, wherein the measuring comprises an immunoassay.
F.11. The method of any of the previous and/or subsequent embodiments, wherein the measuring comprises flow cytometry or mass spectrometry or liquid chromatography tandem mass spectrometry (LC-MS/MS).
F.12. The method of any one of the previous and/or subsequent embodiments, wherein at least 5 of the biomarkers are measured.
G.1. The methods, composition, kit or system of any of the previous and/or subsequent embodiments, wherein at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the biomarkers are measured.
G.2. The methods, compositions, kit or system of any of the previous and/or subsequent embodiments, wherein the measurement may include measurement of at least one normalization (e.g., housekeeping) gene.

References and citations to other documents, such as patents, patent applications, patent publications, journals, books, papers, web contents, have been made throughout this disclosure. All such documents are hereby incorporated herein by reference in their entirety for all purposes. Various modifications and equivalents of those described herein, will become apparent to those skilled in the art from the full contents of this document, including references to the scientific and patent literature cited herein. The subject matter herein contains information, exemplification and guidance that can be adapted to the practice of this disclosure in its various embodiments and equivalents thereof.

That which is claimed is:

1. A method to detect a biomarker associated with kidney fibrosis in an individual comprising the steps of:
obtaining a sample from the individual; and
measuring the amount of the biomarker, and/or the amount of or a mutation in a nucleic acid that encodes for or regulates expression of the biomarker, in the biological sample, wherein the biomarker comprises at least one of one of transforming growth factor beta 1 (TGFB1) or metallopeptidase 2 (MMP2).

2. The method of claim 1, wherein the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7).

3. The method of claim 1, wherein, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2).

4. The method of claim 1, wherein the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2), nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA).

5. The method of claim 1, wherein the measuring comprises measurement of protein, an immunoassay, flow cytometry, mass spectrometry, or analysis of nucleic acid sequence or expression.

6. The method of claim 1, wherein the biological sample comprises a liquid or tissue biopsy, cell-free nucleic acid, blood, urine, serum or plasma.

7. A method to detect the presence of, or susceptibility to, kidney fibrosis in an individual comprising:
obtaining a biological sample from the individual;
measuring the amount of the biomarker, and/or the amount of or a mutation in a nucleic acid (e.g. genomic DNA or mRNA) that encodes for, or regulates expression of a biomarker in the biological sample; and
comparing the amount of, and/or the amount or a mutation in a nucleic acid that encodes for, or regulates expression of the biomarker in the biological sample with a control value for the biomarker.

8. The method of claim 7, wherein the biomarker comprises at least one of transforming growth factor beta 1 (TGFB1) or metallopeptidase 2 (MMP2).

9. The method of claim 7, wherein the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7).

10. The method of claim 7, wherein, the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2)

11. The method of claim 7, wherein the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2), nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA).

12. The method of claim 7, wherein the measuring comprises measurement of protein, an immunoassay, flow cytometry, mass spectrometry, or analysis of nucleic acid sequence or expression.

13. The method of claim 7, wherein the sample comprises a liquid or tissue biopsy, cell-free nucleic acid, blood, urine, serum or plasma.

14. A composition to detect a biomarker associated with kidney fibrosis in an individual comprising reagents that measure the amount of the biomarker, and/or the amount or a mutation in a nucleic acid (e.g. genomic DNA or mRNA) that encodes for, or regulates expression of the biomarker.

15. The composition of claim 14, wherein the biomarker comprises at least one of transforming growth factor beta 1 (TGFB1) or metallopeptidase 2 (MMP2).

16. The composition of claim 14, wherein the biomarker comprises at least one of transforming growth factor beta 1 (TGFB1), metallopeptidase 2 (MMP2), uromodulin (UMOD), SMAD family member 2 (SMAD2), or SMAD family member 7 (SMAD7).

17. The composition of claim 14, wherein the biomarker comprises at least one of transforming growth factor beta 1 (TGFB1), metallopeptidase 2 (MMP2), uromodulin (UMOD), SMAD family member 2 (SMAD2), SMAD family member 7 (SMAD7), connective tissue growth factor (CTGF), or C—C motif chemokine ligand 2 (CCL2).

18. The composition of claim 14, wherein the biomarker comprises at least one of corticosterone, aldosterone, ADAM metallopeptidase domain 17 (ADAM17), C—C motif chemokine ligand 2 (CCL2), cadherin 1 (CDH1), connective tissue growth factor (CTGF), epidermal growth factor receptor (EGFR), fibronectin 1 (FN1), galectin-1 (LGALS1), galectin-3 (LGAS3), hepatocyte growth factor (HGF), intercellular adhesion molecule 1 (ICAM1), interleukin 1 beta (IL1B), interleukin 6 (IL6), Kruppel like factor 15 (KLF15), matrix metallopeptidase 2 (MMP2), matrix metallopeptidase 7 (MMP7), matrix metallopeptidase 9 (MMP9), nuclear factor kappa B subunit 1 (NFKB1), nephrin (NPHS1), podicin (NPHS2), nuclear receptor subfamily 3 group C member 2 (NR3C2), serpin family E member 1 (SERPINE1), SMAD family member 2 (SMAD2), SMAD family member 3 (SMAD3), SMAD family member 4 (SMAD4), SMAD family member 7 (SMAD7), signal transducer and activator of transcription 3 (STAT3), transforming growth factor beta 1 (TGFB1), uromodulin (UMOD), or vascular endothelial growth factor A (VEGFA).

19. The composition of claim 14, wherein the measuring comprises measurement of protein, an immunoassay, flow cytometry, mass spectrometry, or analysis of nucleic acid sequence or expression.

20. The composition of claim 14, wherein the sample comprises a liquid or tissue biopsy, cell-free nucleic acid, blood, urine, serum or plasma.

21. The composition of claim 14, wherein at least one of the reagents is labeled.

22. A kit comprising reagents to detect a biomarker associated with kidney fibrosis in an individual comprising: reagents that measure the amount of the biomarker, and/or the amount of, or a mutation in, a nucleic acid (e.g. genomic DNA or mRNA) that encodes for, or regulates expression of the biomarker; and instructions for use.

23. A system to detect a biomarker associated with kidney fibrosis in an individual comprising:
a station for providing a sample believed to contain the biomarker;
optionally, a station for separating the biomarker from other components in the sample;
a station for measuring the amount of the biomarker; and
a station to analyze the results to determine the presence or amount of the biomarker in the sample.

24. A method of treating an individual having or susceptible to developing kidney fibrosis kidney fibrosis comprising:
obtaining a sample from the individual;
measuring the amount of the biomarker, and/or the amount of or a mutation in a nucleic acid that encodes for, or regulates expression of a biomarker in the sample;
comparing the amount of the biomarker, and/or the amount or a mutation in a nucleic acid that encodes for, or regulates expression of expression of the biomarker, in the sample with a control value the biomarker; and
treating the individual for kidney fibrosis or to reduce the rate of developing kidney fibrosis when a difference between gene expression in the individual and the control value indicates that the individual may have, or is susceptible to developing kidney fibrosis.
